(19) United States
(12) Patent Application Publication
(10) Pub. No.: US 2002/0111500 A1
(43) Pub. Date: Aug. 15, 2002

(54) Preparation of 4-hydroxy-3[2H]-furanones

(76) Inventors: Sergey A. Selifonov, Plymouth, MN (US); Lisa M. Newman, Sunnyvale, CA (US)

Correspondence Address: LAW OFFICES OF JONATHAN ALAN QUINE, P O BOX 458, ALAMEDA, CA, 94501

(21) Appl. No.: 09/870,934

(22) Filed: May 30, 2001

Related U.S. Application Data
(60) Provisional application No. 60/208,375, filed on May 31, 2000.
(60) Provisional application No. 60/261,524, filed on Jan. 12, 2001.

(51) Int. Cl. C12P 17/04; C 07D 3 7/20

(52) U.S. Cl. 549/477; 435/126

(57) ABSTRACT

Biocatalysis is used to prepare 4-hydroxy-3[2H]-furanones from substituted benzenes. A substituted benzene is enzymatically oxidized to form a diol-diene compound, which is then oxidized and cyclized to form a 4-hydroxy-3[2H]-furanone. Dioxygenases are used to perform the enzymatic oxidation. In addition, methods of obtaining improved dioxygenases are provided. Compositions including one or more of the intermediate compounds in the biocatalysis method, the resulting 4-hydroxy-3[2H]-furanone compounds, and improved enzymes are also provided.

### CROSS-REFERENCE TO RELATED APPLICATIONS

Pursuant to 35 U.S.C. §119(e) and any other applicable statute or rule, the present application claims benefit of and priority to U.S. Ser. No. 60/261,524 “Preparation of 4-Hydroxy-3[2H]-Furanones,” by Selifonov et al., filed Jan. 12, 2001; U.S. Ser. No. 60/208,375 “Preparation of 4-Hydroxy-3[2H]-Furanones,” by Selifonov et al., filed May 31, 2000; and co-filed PCT application, “Preparation of 4-Hydroxy-3[2H]-Furanones,” by Selifonov et al., filed May 30, 2001, Attorney Docket No. 02-102920PC.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

4-Hydroxy-2,5-dimethyl-2,3-dihydrofuran-3-one (“strawberry furanone”) is an essential component of strawberry and pineapple aromas that is widely used in the flavor industry. A plurality of synthetic processes for making this furanone and related furanone flavoring compounds are known in the art.

U.S. Pat. No. 2,936,308 describes a reaction of L-rhamnose and piperidine acetate in ethanol to give strawberry furanone in 26% yield. Carbohydrate-based methods for making furanones have been described in U.S. Pat. No. 5,149,840, “Hydroxy Furanone Preparation” by Decnop et al., and in the art cited therein, wherein preparation of strawberry furanone and of 4-hydroxy-5-methyl-2,3-dihydrofuran-3-one from 6-deoxyhexose or from a pentose is achieved by heating the carbohydrates with an amino acid followed by distillation under reduced pressure in the presence of suitable solvent.

Preparation and uses of 4-hydroxy-5-methyl-2,3-dihydrofuran-3-one, a compound with a pleasant maltol-like flavor are described in U.S. Pat. No. 4,013,800, “4-Hydroxy-5-Methyl-2,3-Dihydrofuran-3-one and Methods of Making and Using the Same” by Shimazaki et al. Maillard reactions between xylose or another pentose and one or more amino acids are used to prepare the furanone of interest.

U.S. Pat. No. 4,480,111, “Process for the Preparation of 4-hydroxy-2,5-dimethyl-2,3-dihydrofuran-3-one” by Whitesides et al., describes a method for making strawberry furanone by hydrogenolysis of alkali or alkaline earth metal derivatives of fructose-1,6-diphosphate or fructose 1- or fructose-6-monophosphate in the presence of a metal catalyst.

An alternative method for preparation of strawberry furanone is based on catalytic cyclization of hexane-3,4-diol-2,5-dione that has been described in U.S. Pat. No. 3,694,466, “Process for the Preparation of 2,5-dimethyl-4,5-dihydrofuran-3-ol-4-one” by Buchi et al. The same patent describes methods for preparation of a hexane-3,4-diol-2,5-dione intermediate which are based on reduction of pyruvaldehyde, oxidation of 2,4-dimethyl-2,5-dimethoxy-3,4-dihydrofuran, or oxidation of acetol.

U.S. Pat. No. 4,290,960, “Preparation of 2,5-dimethyl-4-hydroxy-2,3-dihydrofuran-3-one” by Ross et al. describes yet another method for making strawberry furanone via 3,4-epoxy-hexane-2,5-diol.

Other methods of making strawberry furanone use hex-3-yne-2,5-diol, which is described in U.S. Pat. No. 5,580,996, “Oxygen-Containing Aliphatic Compounds and Their Use as Intermediates for the Preparation of 4-Hydroxy-2,5-Dimethyl-3(2H)-Furanone” by Mimoum et al., and in the references cited therein. In these processes, oxidation of the alkyne bond of hex-3-yne-2,5-diol, or its 2,5-ditertbutyloxy- or 2,5-diisoamyloxy-derivatives provides intermediates suitable for cyclization to form the desired furanone.

These processes for making furanone compounds are laborious and products obtained often may not have satisfactory storage stability and/or flavoring properties due to the presence of impurities and by-products. Another major drawback of the existing chemical routes to strawberry furanone, and to related 4-hydroxy-2,3-dihydrofuran-3-one derivatives, is the high manufacturing cost, e.g., the high cost of raw materials.

New or improved methods of making furanone compounds are accordingly desirable, particularly those that take advantage of low cost starting materials, are amenable to industrial manufacturing techniques, and/or produce furanones having desirable flavoring properties and purity levels. The present invention fulfills these and other needs that will become apparent upon complete review of this disclosure.

### SUMMARY OF THE INVENTION

The present invention provides methods of making 4-hydroxy-3[2H]-furanones. In general, the methods involve a combination of biocatalysis steps and chemical synthesis steps. Typically, substituted benzenes, e.g., p-xylene, are enzymatically oxidized to form diol-diene compounds, which are then chemically oxidized to form diol-dione compounds. The diol-dione compounds are cyclized to make 4-hydroxy-3[2H]-furanones. In addition, the invention provides compositions involved in the synthesis of 4-hydroxy-3[2H]-furanones.

In one aspect, the methods of making a 4-hydroxy-3[2H]-furanone comprise providing a substituted benzene and enzymatically oxidizing it, thereby producing a cis-diol-diene compound. The diol-diene compound is oxidized to form a cis-diol-dione compound, which is cyclized to form a 4-hydroxy-3[2H]-furanone.

Typical furanones include, but are not limited to 4-hydroxy-2,5-dimethyl-3[2H]-furanone and other 2,5-substituted 4-hydroxy-3[2H]-furanones, such as those having Formula (2):

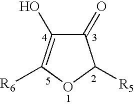

wherein R₅ and R₆ are independently selected from: hydrogen, lower alkyl, methyl, ethyl, propyl, isopropyl, isobutyl, sec-butyl, tert-butyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl. Typically, R₅ and R₆ are not both hydrogen. In some embodiments, R₅ is hydrogen and R₆ is selected from: lower alkyl, e.g., an alkyl comprising about 1 to about 10 carbon atoms, isopropyl, isobutyl, sec-butyl, tert-butyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, and geminal dialkoxyalkyl; or R₅ is methyl and R₆ is selected from: ethyl, propyl, isopropyl, acetyl, propanoyl, 1-hydroxyethyl, and 2-hydroxyethyl; or R₅ is ethyl and R₆ is selected from ethyl, acetyl, and propanoyl. Typically R₅ and R₆ are both methyl groups or R₅ and R₆ are different and at least one of them comprises two or more carbon atoms.

Typically, enzymatic oxidation of a substituted benzene produces a diol-diene compound having Formula (5):

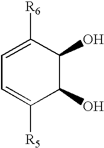

wherein R₅ and R₆ are defined as above. The diol-diene compound is optionally a symmetrical achiral diol-diene or a chiral cis-diol-diene compound. For example, when p-xylene is used as a starting material, enzymatic oxidation produces cis-1,2-dihydroxy-3,6-dimethylhexa-3,5-diene.

In one embodiment, enzymatic oxidation comprises contacting a substituted benzene with a dioxygenase, e.g., an arene dioxygenase, or one or more cells, e.g., microbial or bacterial cells, which possess dioxygenase activity. In some embodiments, the substituted benzene is contacted with one or more dioxygenase in the presence of water and/or an organic solvent.

Typical dioxygenases include, but are not limited to, toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene 1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, and ring dihydroxylating dioxygenase. These dioxygenases are optionally encoded by a nucleic acid comprising a mutant or chimeric dioxygenase or arene dioxygenase nucleotide sequence.

In other embodiments, the dioxygenase used to oxidize a substituted benzene is encoded by a nucleic acid comprising at least 60 contiguous nucleotides of a nucleic acid encoding any of the above enzymes or any dioxygenase or arene dioxygenase that is present in a public database such as GenBank™ at the time of filing of the subject application; a nucleic acid that encodes a polypeptide having at least 20 contiguous amino acids of one or more of the above enzymes; or a nucleic acid that hybridizes under stringent conditions to any of the above nucleic acids.

After enzymatic oxidation, the diol-diene is typically chemically oxidized to form a diol-dione compound, e.g., a cis-diol-dione, having Formula (7):

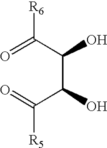

wherein R₅ and R₆ are defined as above. For example, the diol-dione compound formed optionally comprises hexane-3,4-cis-diol-2,5-dione.

In one embodiment, the diol-diene compound is oxidized in a substantially aqueous solvent comprising ozone or a mixture of ozone and oxygen in the presence of boric acid, arylboronic acid, alkyl boronic acid, or a metal salt thereof. In some embodiments, the diol-diene compound is attached to a resin or inorganic adsorbent material, e.g., a material comprising an alkylboronate moiety or an arylboronate moiety.

In another embodiment, oxidation of the diol-diene compound involves protection of the diol groups before oxidation and deprotection after oxidation. The two hydroxyl groups of the diol-diene compound are protected, thereby producing a protected diol-diene compound, which is then oxidized to form a protected dione compound, e.g., a symmetrical achiral dione compound. The protected diol-dione compound is then deprotected to provide the diol-dione compound.

Protecting groups of use in the present invention include, but are not limited to, cyclic ketals, cyclic acetals, ether groups, and ester groups. Contacting a diol-diene of the invention with one or more ketone or ketal, e.g., in the presence of a catalyst, results in a cyclic ketal or a cyclic acetal having Formula (9):

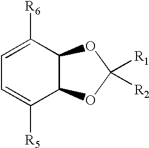

wherein R₅ and R₆ are defined as described above and R₁ and R₂ are each independently selected from: hydrogen, alkyl, aryl, and aralkyl or R₁ and R₂ together comprise a cycloalkyl ring, which cycloalkyl ring comprises about 5 to about 6 carbon atoms. R₁ and R₂ optionally comprise the same or different groups. Typically at least one of R₁ and R₂ is not hydrogen.

Various catalysts are optionally used to form protected diol-dione compounds. In some embodiments, an acid catalyst is optionally used to form a compound having Formula (9). For example, aryl or alkylsulfonic acid; a solid phase catalyst, e.g., a solid phase acid; or a resin, such as a resin comprising one or more protonated sulfonic groups, optionally serves as a catalyst in the present invention.

Using an ether group or an ester group as the protecting group results in a compound having Formula (11):

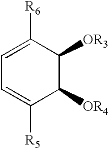

wherein R₅ and R₆ are defined as above and R₃ and R₄ are independently selected from: hydrogen, alkylacyl, arylacyl, tert-butyl, trialkylsilyl, and aralkylacyl, or R₃ and R₄ together comprise a boron moiety comprising an alkyl, aryl, or hydroxy substituent, e.g., an alkylboronate or arylboronate moiety.

A protected diol-diene compound is optionally contacted with one or more oxidizing reagent to provide a protected dione compound having Formula (14):

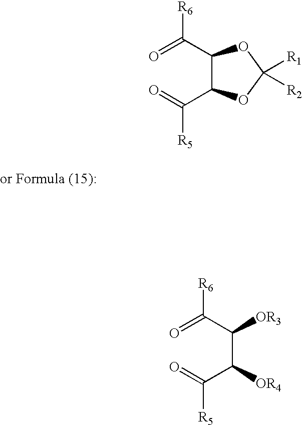

wherein R₁, R₂, R₃, R₄, R₅, and R₆ are defined as above. Oxidizing reagents include, but are not limited to, an alkali metal salt, an alkali metal permanganate salt, an alkali metal periodate salt, an alkali metal hypochlorite salt, an organic peroxyacid, an organic peroxide, an inorganic peroxyacid, an inorganic peroxide, ozone, and an ozone/oxygen mixture. In some embodiments, the protected diol-diene compound is optionally contacted with an alkali metal hypochlorite salt in the presence of catalytic amounts of ruthenium halide or oxide.

The protected dione compound is then typically contacted with one or more deprotecting reagent. For example, when the protected dione compound comprises a cyclic ketal or a cyclic acetal, the one or more deprotecting reagent optionally comprises acetic acid, hydrochloric acid, sulfuric acid, phosphoric acid, oxalic acid, or citric acid. Deprotection typically results in a diol-dione compound having Formula (17):

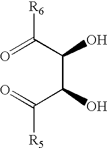

wherein R₅ and R₆ are defined as provided above. The diol-dione compound, e.g., a cis-diol-dione compound, is optionally isolated. In other embodiments, oxidizing the diol-diene and cyclizing the resulting diol-dione are optionally performed contemporaneously with the cyclization of the diol-dione compound performed on an unisolated diol-dione compound.

Cyclization of the diol-dione compound typically occurs in the presence of a catalyst or an amino acid. Typical catalysts include, but are not limited to, an alkali metal or alkali earth metal salt of a dibasic or tribasic acid.

In another aspect, the present invention provides compositions comprising a compound having Formula (14), Formula (15), or Formula (17) as described above. The compositions typically comprise substantially all cis-stereoisomers.

In another aspect, the invention provides compositions comprising Formula (2) as described above, e.g., at least 0.1 ppm of one or more compounds having Formula (2). The compositions typically comprise a food flavoring composition, a beverage flavoring composition, an odor control composition, a laundry composition, or the like.

In another aspect, the present invention provides methods of producing enzymes to oxidize substituted benzenes as described above. The method comprises providing a population of DNA fragments encoding at least one parental enzyme that oxidizes a substituted benzene. The parental enzyme is typically selected from those provided above. The DNA fragments are recombined to produce a library of recombinant DNA segments and screened to identify DNA segments that encode an artificially evolved enzyme with greater oxidizing activity, e.g., higher conversion rate or broader substrate specificity, for substituted benzenes than that encoded by the parental enzyme. These steps are optionally repeated one or more times to produce more recombinant nucleic acids.

In other aspects, the present invention provides nucleic acids and nucleic acid libraries produced by the above method, cell populations comprising such nucleic acids and/or libraries, and compositions comprising enzymes produced as described above and one or more substituted benzene as described above.

### BRIEF DESCRIPTION OF THE FIGURES

FIG. 1: Schematic drawing illustrating oxidation of a diol-diene compound using a boronate resin.

FIG. 2: Schematic illustration of an oxidation reaction comprising epoxidation of protected diol-diene compounds.

FIG. 3: Schematic illustration of an oxidation reaction involving epoxidation of unprotected diol-diene compounds.

FIG. 4: Equilibrium between free diones (protected or unprotected diol-dione compounds) and cyclic pseudofuranose ketals.

FIG. 5: Furanone tautomers of a compound having Formula (1) or (2).

### DETAILED DISCUSSION OF THE INVENTION

The present invention provides methods for the preparation of furanones, e.g., 4-hydroxy-3[2H]-furanones, in particular, 4-hydroxy-2,5-dimethyl-2,3-dihydrofuran-3-one. In general, biocatalytic oxidation is used to transform a substituted benzene to a glycol compound, e.g., a cis-diol-diene compound. This is followed by chemical reactions to oxidize the diol-diene compound to a diol-dione compound, which is then cyclized to produce a 4-hydroxy-3[2H]-furanone, such as 4-hydroxy-2,5-dimethyl-2,3-dihydrofuran-3-one. Compositions comprising furanones and intermediates obtained from the preparation methods described above are also provided. In addition, the present invention provides methods for producing improved enzymes to catalyze the biocatalytic oxidation.

Chemical Structure Definitions

As used herein, “furanone” refers to a class of compounds generally referred to as 4-hydroxy-3[2H]-furanones. A preferred furanone is 4-hydroxy-2,5-dimethyl-2,3-dihydrofuran-3-one, having Formula (1):

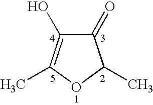

Other furanone compounds of interest comprise compounds having Formula (2):

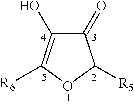

wherein R₅ and R₆ are independently selected from: hydrogen, lower alkyl, methyl, ethyl, propyl, isopropyl, isobutyl, sec-butyl, tert-butyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl. In some embodiments, R₅ is hydrogen and R₆ is selected from: lower alkyl, e.g., an alkyl comprising about 1 to about 10 carbon atoms or more typically about 1 to about 6 carbons, isopropyl, isobutyl, sec-butyl, tert-butyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, and geminal dialkoxyalkyl. Typically, R₅ and R₆ are not both hydrogen. In other embodiments, R₅ is methyl and R₆ is selected from: ethyl, propyl, isopropyl, acetyl, propanoyl, 1-hydroxyethyl, and 2-hydroxyethyl. Alternatively, R₅ is ethyl and R₆ is selected from ethyl, acetyl, and propanoyl. Typically R₅ and R₆ are both methyl groups; or R₅ and R₆ are different and at least one of them comprises two or more carbon atoms. These compositions and the methods of making them are features of the invention.

Formula (3) is used herein to refer to a compound having the following formula:

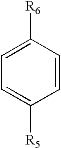

wherein R₅ and R₆ are defined as above. Compounds such as those of Formula (3) are generally referred to as “substituted benzenes.” A particular substituted benzene of interest in the present application is p-xylene, having the following formula:

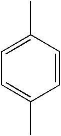

Formula (4), as used herein, refers to compounds having the formula:

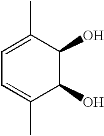

and Formula (5) refers to compounds having the formula:

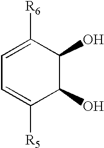

wherein R₅ and R₆ are defined as described above. The compounds of Formulas (4) and (5) are referred to as “diol-diene” compounds or glycol compounds. The compound of Formula (4) is typically known as 1,2-dihydroxy-3,6-dimethylhexa-3,5-diene. In the present invention, these compounds typically comprise substantially all cis-stereoisomers, e.g., typically over 95%, more typically over 99% cis-stereoisomers. When R₅ and R₆ are the same substituent, the diol-diene compounds of the invention comprise symmetrical achiral diol-dienes. Alternatively, chiral diol-dienes are formed when R₅ and R₆ comprise different substituents.

Formula (6) as used herein, refers to a compound having the formula:

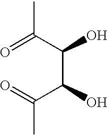

and Formula (7) refers to compounds having the formula:

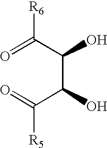

wherein R₅ and R₆ are defined as described above. The compounds represented by Formula (6) and Formula (7) are typically referred to as diol-dione compounds. Typical diol-diones of the present invention comprise cis-diol-dione compounds, such as hexane-3,4-cis-diol-2,5-dione, which is represented by Formula (6). These compounds and the methods of making them are a feature of the present invention.

Formula (8) is used herein to refer to compounds having the formula:

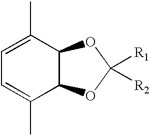

and Formula (9) refers to compounds having the formula:

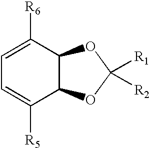

wherein R₅ and R₆ are defined as described above and R₁ and R₂ are each independently selected from: hydrogen, alkyl, aryl, and aralkyl; or R₁ and R₂ together comprise a cycloalkyl ring comprising about 5 to about 6 carbon atoms. R₁ and R₂ optionally comprise the same or different substituents. Typically, at least one of R₁ and R₂ is not hydrogen.

Formula (10) refers to compounds having the formula:

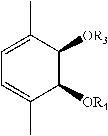

and Formula (11) refers to compounds having the formula:

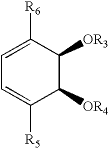

wherein R₅ and R₆ are defined as described above and R₃ and R₄ are independently selected from: hydrogen, alkylacyl, arylacyl, tert-butyl, trialkylsilyl, and aralkylacyl. In some embodiments, R₃ and R₄ together comprise a boron moiety having an alkyl, aryl or hydroxy substituent, e.g., an alkylboronate or arylboronate moiety.

The compounds of Formula (8), (9), (10), and (11) are referred to herein as “protected diol-dienes” or “protected cis-diol-dienes.” These compounds are typically formed when a protecting group is added to a compound having Formula (4) or (5). Typical protecting groups used in the present invention form cyclic ketals or cyclic acetals, as shown in Formulas(8) and (9), ether groups or ester groups, as shown in Formulas(10) and (11), or the like, when added to compounds of Formulas (4) and (5).

Formula (12), as used herein, refers to compounds having the formula:

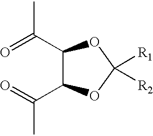

Formula (13) refers to compounds having the formula:

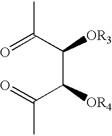

Formula (14) refers to compounds having the formula:

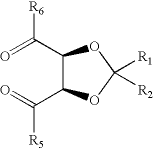

and Formula (15) refers to compounds having the formula:

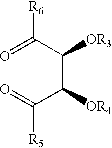

In Formulas (12), (13), (14), and (15), the R groups, e.g., R₁, R₂, R₃, R₄, R₅, and R₆, are all defined as described above. These compounds are referred to herein as “protected dione compounds.” These compounds are typically deprotected to form diol-dione compounds as represented by Formulas (6) and (7). The present invention provides methods of making and using the above compounds, e.g., to form 4-hydroxy-3[2H]-furanone compounds, as well as compositions comprising the compounds.

I. Introduction

The furanone compound, 4-hydroxy-2,5-dimethyl-2,3-dihydrofuran-3-one, a 4-hydroxy-3[2H]-furanone compound as represented by Formula (1), is an essential component of strawberry and pineapple aromas. As such, it is widely used in the flavor industry. Related compounds include other furanones, such as 4-hydroxy-5-methyl-2,3-dihydrofuran-3-one, 4-hydroxy-2,5-ethyl-2,3-dihydrofuran-3-one. Other 2- and/or 5-substituted 4-hydroxy-2,3-dihydrofuran-3-ones, e.g., those compounds having Formula (2), are also useful in flavoring compositions. The present invention provides an inexpensive method of preparing 4-hydroxy-2,5-dimethyl-2,3-dihydrofuran-3-one and other related furanones from abundant raw materials, such as p-xylene and corresponding alkyl-substituted aromatic compounds. These compounds are referred to herein as substituted benzenes and are represented by Formula (3).

In general, the invention uses biocatalysis-based methods of making oxygen-containing aliphatic compounds, which are optionally converted to the furanones of interest. In addition, the invention describes suitable arene dioxygenase enzymes, genes, and microorganisms and methods for their improvement and use in the first step of furanone synthesis, e.g., whole-cell dihydroxylation of substituted benzenes, e.g., p-xylene and related compounds, to symmetrical achiral or chiral cis-glycol compounds, e.g., diol-diene compounds as described above. Chemical synthesis is typically used to convert the cis-glycol compounds into the furanones of interest, e.g., by oxidizing the glycol compound to form a diol-dione and cyclizing the dione compound to form a furanone ring structure.

The method of preparation begins by reacting a substituted benzene, such as p-xylene, with oxygen, in the presence of microbial cells possessing enzymatic activity of at least one type of dioxygenase, e.g., arene dioxygenase, that is capable of catalyzing oxidation of substituted benzenes such as p-xylene and those of Formula (3). The oxidation results in a glycol compound, typically a cis-glycol compound, which is symmetrical when p-xylene is the starting compound or when R₅ and R₆ are the same in Formula (3). Formula (5) represents a typical compound resulting from the enzymatic oxidation of substituted benzenes. Formula (4) represents the resulting compound when p-xylene is enzymatically oxidized, e.g., by a dioxygenase.

The oxidized compounds are typically referred to as diol-diene compounds or glycol compounds, which are then oxidized to form diol-dione compounds. Typically, the diol groups are protected before oxidation of the hexa-diene ring structure to form a dione. The diol-diene compounds are typically protected using cyclic ketals or cyclic acetals as represented by Formulas (8) and (9) or with ester or ether groups, as shown by Formulas (10) and (11). The protected compounds are oxidized using a suitable oxidizing reagent to provide the corresponding protected dione compound. See, e.g., Formulas (12), (13), (14), and (15). The protected diones are deprotected to remove the hydroxyl protecting groups and provide a diol-dione compound such as hexane-3,4-cis-diol-2,5-dione. Formulas (6) and (7) represent diol-dione compounds.

The diol-dione compounds are then cyclized, e.g., in the presence of a suitable catalyst to provide a furanone. For example, hexane-3,4-cis-diol-2,5-dione is cyclized to form 4-hydroxy-2,5-dimethyl-2,3-dihydrofuran-3-one. The above steps for preparing furanones are described in more detail below along with methods of making improved enzymes for use in the preparation.

II. Enzymatic Oxidation of Substituted Benzenes to Form Diol-diene Compounds

The first step in the preparation of 4-hydroxy-3[2H]-furanones, e.g., 4-hydroxy-2,5-dimethyl-2,3-dihydrofuran-3-one comprises the enzymatic oxidation of substituted benzenes to form diol-dione compounds. Substituted benzenes of interest in the present invention include, but are not limited to, those described above, e.g., p-xylene, and compounds having Formula (3). Diol-diene compounds produced in this step are also described above, e.g., compounds having Formula (4) or (5). These compounds are typically cis-diol-diene or vicinal cis-diol-diene compounds. For example, in compounds having Formula (5), the hydroxyl groups are typically vicinal and the relative configuration of the hydroxyl groups is cis- and the absolute configuration is R or S. When R₅ and R₆ are different, the compounds are chiral compounds, with an enantiomeric excess anywhere in the range of 0% to about 100%.

p-Xylene and other arene oxidations are conveniently carried out using cells, e.g., microbial or bacterial cells, that possess sufficient activity of one or more dioxygenases, e.g., arene dioxygenases, that act on arenes as substrates. Oxidation of para-xylene to symmetrical (achiral) cis-1,2-dihydroxy-3,6-dimethylhexa-3,5-diene was first described by Gibson and co-workers (J. Bacteriol., 1974, 119(3):930-936), who studied initial reactions and mechanisms involved in bacterial degradation of aromatic hydrocarbons. The cis-diol compound obtained from p-xylene was obtained in low yield (189 mg/L) by using a mutant 39/D strain of Pseudomonas putida F1 that lacks cis-dihydrodiol dehydrogenase activity.

Various microorganisms are optionally used to oxidize the arene of interest with a suitable dioxygenase, including, but not limited to, bacteria, cyanobacteria, fungi, yeasts, and the like. A preferred embodiment uses bacterial strains. Various bacterial strains are optionally used for the purpose, including E.coli and other species selected from the following non-limiting examples of genera of known microorganisms: Pseudomonas, Rhodococcus, Burkholderia, Sphingomonas, Comamonas, Alcaligenes, Acinetobacter, Bacillus, and the like. E.coli is typically used because this organism is generally recognized as safe in biotechnological applications. Other non-pathogenic species are also optionally used. The strains are optionally prototrophic or auxotrophic in respect to different growth requirements and nutrients, and the bacterial cells can be grown in a variety of media of defined or undefined compositions well known in the art. Various carbon and nitrogen sources are optionally used. A typical principal nitrogen source used comprises ammonia. Preferred principal carbon sources for E.coli include, but are not limited to, glucose, glycerol, ethanol, lactate, succinate, fumarate, amino acids, acetate, and the like. For growth in defined and non-defined media, supplements of trace minerals are known in the art. Supplements comprising iron (II) salts are preferred.

One attribute of microorganisms useful for effecting the formation of diol-diene compounds from aromatic substrates, e.g., substituted benzenes, is the sufficient activity of one or more dioxygenase or arene dioxygenases. Dioxygenases act on aromatic compounds as substrates, bringing about dihydroxylation of the compounds to diol-diene compounds. The organisms used to generate the diol-diene compounds, e.g., those of Formulas (4) and (5), typically substantially lack arene cis-dihydrodiol dehydrogenase activity, an enzyme normally involved in subsequent reaction of bacterial catabolism of aromatic compounds. An example of a suitable microorganism is the mutant strain of Pseudomonas putida F1/39D (ATCC No. 700008) which possesses inducible activity of toluene dioxygenase and lacks activity of toluene cis-dihydrodiol dehydrogenase.

Preferred microorganisms for effecting the oxidation of substituted benzenes typically do so both rapidly and in high concentrations and are suitable for large-scale industrial applications. Many methods are known in the art that allow for improvement of activity of desired enzymes in microbial cells. Such methods include microbial strain engineering methods. For example, microbial engineering optionally provides for incorporation of multiple copies of complete gene sets, encoding multi-component enzymes and/or genes encoding individual subunits, on a plasmid and/or on the chromosome. In other embodiments, a desired gene(s) is placed under promoters of various strength and host specificity, e.g., to attain desired levels of enzyme expression. Various other methods provide for sequence modification of the gene(s) to alter or improve desired catalytic properties of the enzyme(s).

Many arene dioxygenases suitable for practicing the present invention are known in the art and are also amenable to techniques used for strain engineering and improvement. Bacterial arene dioxygenases, e.g. toluene dioxygenase, naphthalene dioxygenase, and the like, are known in the art as enzymes that effect the reductive dioxygenation of aromatic compounds (Zylstra & Gibson D. T. 1991. Aromatic hydrocarbon degradation. A molecular approach. Genetic Engineering, ed. by J. K. Setlow. Plenum Press, NY, v.13:183-203 ), and hence they are useful catalysts that provide for the biocatalytic preparation of cis-dihydrodiols from a variety of aromatic compounds. Organisms possessing arene dioxygenase (cis-dihydroxylating) activity are well known in the art, and many genes encoding dioxygenases with varying catalytic properties and substrate specificity have been described. See, e.g., the enzymes listed in Table 1. Chiral arene cis-dihydrodiols, generated by dioxygenases from aromatic substrates, are also well known in the art as useful starting materials to prepare a variety of oxygen-containing cyclic and acyclic compounds by means of various oxidation and addition reactions. Synthetic utility of arene cis-dihydrodiols has been comprehensively reviewed, e.g., in Brown S. M., and Hudlicky T., 1993, Organic Synthesis: Theory and Applications, ed. T. Hudlicky, JAI Press Inc., Greenwich, Conn., London, England, vol. 2, p. 113-176.

Arene dioxygenases are known in the art as multi-component enzymes typically comprising about 2 to about 4 types of subunits having different functions in the catalytic process. It is also known in the art that artificial functional arene dioxygenases are optionally constructed and expressed as chimerical sets of subunits recruited from more than one set of genes encoding wild-type arene dioxygenases from the same source microorganism, or from multiple sources.

Examples of suitable arene dioxygenase genes (whether complete sets encoding all needed subunits, or genes encoding individual subunits) that are optionally expressed and/or altered to effect and improve the parameters of the desired conversion of arenes to diol-dienes, as provided by formulas (4) and (5), include, but are not limited to, the following genes: toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene (cumene) dioxygenase, biphenyl dioxygenase, and naphthalene dioxygenase. These genes and other suitable genes are provided in Table 1 and referenced by GenBank IDs.

TABLE 1
EXAMPLES OF KNOWN ARENE DIOXYGENASES IN GENBANK

| | GENBANK | SOURCE | ACCESSION | | |
|---|---|---|---|---|---|
| ENZYME NAME | LOCUS | ORGANISM | NUMBER | NID | OTHER GENBANK IDS |
| toluene dioxygenase | PSETODC1C | Pseudomonas putida F1 | J04996 | g151600 | VERSION J04996.1 |
| | | | | | GI:151600 |
| toluene dioxygenase | PPUY18245 | Pseudomonas putida | Y18245 | g4914628 | VERSION Y18245.1 |
| | | | | | GI:4914628 |
| tetrachlorobenzene | BSU78099 | Burkholderia sp. PS12 | U78099 | g3176648 | VERSION U78099.1 |
| dioxygenase | | | | | GI:3176648 |
| 1,2,4-trichlorochlorobenzene | PSU15298 | Pseudomonas sp. | U15298, | g557069 | VERSION U15298.1 |
| dioxygenase | | | M61114 | | GI:557069 |
| 1,2,4-trichloro-benzene | AB019032 | Ralstonia eutropha plasmid | AB019032 | g4210463 | VERSION AB019032 |
| dioxygenase | | pENH91 | | | GI:4210463 |
| ethylbenzene dioxygenase | AF049851 | Pseudomonas fluorescens | AF049851 | g4105708 | VERSION AF049851.1 |
| | | | | | GI:4105708 |
| chlorobenzene dioxygenase | RSP6307 | Ralstonia sp. | AJ006307 | g3184040 | VERSION AJ006307.1 |
| | | | | | GI:3184040 |
| benzene dioxygenase | E08552 | Pseudomonas sp. | E08552 | g2176667 | VERSION E08552.1 |
| | | | | | GI:2176667 |
| benzene dioxygenase | PSEBDO | Pseudomonas putida | M17904 | g151068 | VERSION M17904.1 |
| | | | | | GI:151068 |
| benzene oxygenase | PSEBEDC12A | Pseudomonas putida | L04642 | g474888 | VERSION L04642.1 |
| | AF148496 | | L04643 | | GI:474888 |
| isopropylbenzene dioxygenase | AF006691 | Pseudomonas putida RE204 | AF006691 | g2822263 | VERSION AF006691.1 |
| | | | | | GI:2822263 |
| isopropylbenzene dioxygenase | PJU53507 | Pseudomonas JR1 | U53507 | g1685012 | VERSION U53507.1 |
| | | | | | GI:1685012 |
| isopropylbenzene dioxygenase | PSECUMA | Pseudomonas fluorescens | D37828 | g1256702 | VERSION D37828.1 |
| | | | | | GI:1256702 |
| isopropylbenzene 2,3- | REU24277 | Rhodococcus erythropolis | U24277 | g1542959 | VERSION U24277.1 |
| dioxygenase | | | | | GI:1542959 |
| biphenyl dioxygenase | PSU95054 | Pseudomonas sp. B4 | U95054 | g2687345 | VERSION U95054.1 |
| | | | | | GI:2687345 |
| biphenyl dioxygenase | CTU47637 | Comamonas testosteroni | U47637 | g1245151 | VERSION U47637.1 |
| | | | | | GI:1245151 |
| biphenyl dioxygenase | D88021 | Rhodococcus erythropolis | D88021 | g3059208 | VERSION D88021.1 |
| | | TA421 | | | GI:3059208 |
| biphenyl dioxygenase | D88020 | Rhodococcus erythropolis | D88020 | g3059203 | VERSION D88020.1 |
| | | TA421 | | | GI:3059203 |
| biphenyl dioxygenase | PSEBPHA | Pseudomonas sp. LB400 | M86348 | g349602 | VERSION M86348.1 |
| | | | | | GI:349602 |
| biphenyl dioxygenase | PSEBPHABCC | Pseudomonas sp. | D17319 | g391831 | VERSION D17319.1 |
| | | | | | GI:391831 |
| biphenyl dioxygenase | RERBPHA1 | Rhodococcus sp. | D32142 | g510284 | VERSION D32142.1 |
| | | | | | GI:510284 |
| biphenyl dioxygenase | RSU27591 | Rhodococcus sp. M5 | U27591 | g927231 | VERSION U27591.1 |
| | | | | | GI:927231 |
| biphenyl dioxygenase | RGBPHA | Rhodococcus globerulus P6 | X80041 | g607171 | VERSION X80041.1 |
| | | | | | GI:607171 |
| biphenyl dioxygenase | PSEBPHABC | P. pseudoalcaligenes KF707 | M83673 | g151090 | VERSION M83673.1 |
| | | | | | GI:151090 |
| biphenyl dioxygenase | AF053823 | Synthetic construct | AF053823 | g4377733 | VERSION AF053823.1 |
| | | | | | GI:4377733 |
| biphenyl dioxygenase | AF053824 | Synthetic construct | AF053824 | g4377735 | VERSION AF053824.1 |
| | | | | | GI:4377735 |
| biphenyl dioxygenase | AF053825 | Synthetic construct | AF053825 | g4377737 | VERSION AF053825.1 |
| | | | | | GI:4377737 |
| biphenyl dioxygenase | AF053826 | Synthetic construct | AF053826 | g4377739 | VERSION AF053826.1 |
| | | | | | GI:4377739 |
| biphenyl dioxygenase | AF053827 | Synthetic construct | AF053827 | g4377741 | VERSION AF053827.1 |
| | | | | | GI:4377741 |
| indene 1,2-dioxygenease | AF121905 | Rhodococcus sp. I24 | AF121905 | g4585358 | VERSION AF121905.1 |
| | | | | | GI:4585358 |
| naphthalene dioxygenase | AF061751 | Burkholderia sp. strain RP007 | AF061751 | g3820512 | VERSION AF061751.1 |
| | | | | | GI:3820512 |
| naphthalene dioxygenase | PSENDOABC | Pseudomonas putida | M23914 | g151392 | VERSION M23914.1 |
| | | | | | GI:151392 |
| naphthalene dioxygenase | AF004284 | Pseudomonas putida | AF004284 | g2199561 | VERSION AF004284.1 |
| | | | | | GI:2199561 |
| naphthalene dioxygenase | AF039533 | Pseudomonas stutzeri | AF039533 | g4104750 | VERSION AF039533.1 |
| | | | | | GI:4104750 |
| naphthalene dioxygenase | AF004283 | Pseudomonas fluorescens | AF004283 | g2199557 | VERSION AF004283.1 |
| | | | | | GI:2199557 |
| naphthalene dioxygenase | AF036940 | Pseudomonas sp. U2 plasmid | AF036940 | g4220428 | VERSION AF036940.1 |
| | | pWWU2 | AF081362 | | GI:4220428 |
| naphthalene dioxygenase | AF082663 | Rhodococcus sp. | AF082663 | g4826635 | VERSION AF082663.2 |
| | | NCIMB12038 | | | GI:4826635 |
| naphthalene dioxygenase | PSEORF1 | Pseudomonas aeruginosa | D84146 | g1255665 | VERSION D84146.1 |
| | | | | | GI:1255665 |
| naphthalene dioxygenase | AB004059 | Pseudomonas putida OUS82 | AB004059 | g2189972 | VERSION AB004059.1 |
| | | | D16629 | | GI:2189972 |
| naphthalene dioxygenase | PSENAPDOXA | P. putida | M83949 | g151384 | VERSION M83949.1 |
| | | | | | GI:151384 |
| naphthalene dioxygenase | PSU49496 | Pseudomonas sp. strain 9816- | U49496 | g1224113 | VERSION U49496.1 |
| | | 4 | | | GI:1224113 |
| naphthalene dioxygenase | AF010471 | Pseudomonas putida plasmid | AF010471 | g2246751 | VERSION AF010471.1 |
| | | NPL1 | | | GI:2246751 |
| 2-Nitrotoluene-2,3- | PSU49504 | Pseudomonas sp. | U49504 | g1773273 | VERSION U49504.1 |
| dioxygenase | | | | | GI:1773273 |
| 2,4-Dinitrotoluene dioxygenase | BSU62430 | Burkholderia sp. RASC | U62430 | g1478283 | VERSION U62430.1 |
| | | | | | GI:1478283 |
| phenanthrene dioxygenase | AB024945 | Alcaligenes faecalis | AB024945 | g4586270 | VERSION AB024945.1 |
| | | | | | GI:4586270 |
| phenylpropionate/cinnamate | ECHCAA234 | E. coli | Y11070 | g2072109 | VERSION Y11070.1 |
| 2,3-dioxygenase | | | | | GI:2072109 |
| 2-halobenzoate 1,2- | PCCBDABC | P. cepacia (2CBS) | X79076 | g758208 | VERSION X79076.1 |
| dioxygenase | | | | | GI:758208 |
| ortho-halobenzoate 1,2- | AF121970 | Pseudomonas aeruginosa | AF121970 | g4406503 | VERSION AF121970.1 |
| dioxygenase | | | | | GI:4406503 |
| anthranilate dioxygenase | AF071556 | Acinetobacter sp. ADP1 | AF071556 | g3511231 | VERSION AF071556.1 |
| | | | | | GI:3511231 |
| m,p-toluate 1,2-dioxygenase | PWWXYL | Pseudomonas putida plasmid | M64747 | g151718 | VERSION M64747.1 |
| | | pWW0 | | | GI:151718 |
| p-cumate 2,3-dioxygenase | PPU24215 | Pseudomonas putida F1 | U24215 | g2228230 | VERSION U24215.1 |
| | | | | | GI:2228230 |
| m,p-toluate 1,2 dioxygenase | AF134348 | Pseudomonas putida plasmid | AF134348 | g4877824 | VERSION AF134348.1 |
| | | pDK1 | | | GI:4877824 |
| 3(4)-phenoxybenzoate 3,4- | PPPOBAB | P. pseudoalcaligenes | X78823 | g473249 | VERSION X78823.1 |
| dioxygenase | | (POB310) | | | GI:473249 |
| 3-chlorobenzoate-3,4- | U18133 | Alcaligenes sp. Tn5271 | U18133 | g2073549 | VERSION U18133.1 |
| dioxygenase | | | U00692 | | GI:2073549 |
| phthalate 3,4-dioxygenase | AF095748 | Burkholderia cepacia | AF095748 | g4128211 | VERSION AF095748.1 |
| | | | | | GI:4128211 |
| diterpenoid ring dihydroxylating | AF145210 | Pseudomonas vancouverensis | AF145210 | g5059169 | VERSION AF145210.1 |
| dioxygenase | | strain DhA-51 | | | GI:5059169 |
| diterpenoid ring hydroxylating | AF119621 | Pseudomonas abietaniphila | AF119621 | g4455069 | VERSION AF119621.1 |
| | | | | | GI:4455069 |
| dioxygenase | | BKME-9 | | | |
| aniline 1,2-dioxygenase | ACCANI | Acinetobacter sp. plasmid | D86080 | g1395138 | VERSION D86080.1 |
| | | pYA1 | | | GI:1395138 |
| aniline 1,2-dioxygenase | D85415 | Pseudomonas putida | D85415 | g1841358 | VERSION D85415.1 |
| | | | | | GI:1841358 |
| carbazole dioxygenase | AF060489 | Sphingomonas sp. CB3 | AF060489 | g3243166 | VERSION AF060489.1 |
| | | | | | GI:3243166 |
| carbazole dioxygenase | AB001723 | Pseudomonas stutzeri | AB001723 | g3293057 | VERSION AB001723.1 |
| | | | | | GI:3293057 |
| carbazole 1,9a-dioxygenase | D89064 | Pseudomonas sp. | D89064 | g2317677 | VERSION D89064.1 |
| | | | | | GI:2317677 |
| ring dihydroxylaing | SSU65001 | Sphingomonas sp. | U65001 | g5578702 | VERSION U65001.3 |
| dioxygenase | | | | | GI:5578702 |
| ring dihydroxylating | AF079317 | Sphingomonas | AF079317 | g3378261 | VERSION AF079317.1 |
| dioxygenase | | aromaticivorans plasmid pNL1 | | | GI:3378261 |
| alkylbenzene dioxygenase | PPU293587 | Pseudomonas putida O1G3 | AJ293587 | g9369339 | Version AJ293587.1 |
| | | | | | GI:9369339 |
| phenanthrene dioxygenase | AB031319 | Nocardiodes sp. KP7 | AB031319 | g7619812 | Version AB031319.1 |
| | | | | | GI:7619812 |

Additional preferred examples of arene dioxygenase genes include any mutant or chimerical dioxygenase genes having a polynucleotide sequence incorporating at least one continuous polynucleotide sequence comprising about 60 or more contiguous nucleotides present in a polynucleotide sequence encoding any of the above dioxygenases, the dioxygenases listed in Table 1, or any arene dioxygenase present in a public database, such as GENBANK at the time of filing of the subject application. In addition, nucleic acids that hybridize under stringent conditions to at least one of the above described nucleic acids, e.g., those encoding dioxygenases, are also useful in the present invention for oxidizing substituted benzenes.

“Stringent hybridization conditions” in the context of nucleic acid hybridization experiments such as Southern and northern hybridizations are sequence dependent, and are different under different environmental parameters. An extensive guide to the hybridization of nucleic acids is found in Tijssen (1993), supra. and in Hames and Higgins, 1 and 2. For purposes of the present invention, generally, “highly stringent” hybridization and wash conditions are selected to be about 5° C. lower than the thermal melting point (Tₘ) for the specific sequence at a defined ionic strength and pH. The Tₘ is the temperature (under defined ionic strength and pH) at which 50% of the test sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. In general, a signal to noise ratio of 5× (or higher) than that observed for an unrelated probe in the particular hybridization assay indicates detection of a specific hybridization.

Additional preferred dioxygenases include, but are not limited to, those having polypeptide sequences incorporating at least one continuous polypeptide sequence comprising about 20 or more contiguous amino acid residues present in a polypeptide sequence of any of the above dioxygenases, the dioxygenases listed in Table 1, or any arene dioxygenase present in a public database, such as GENBANK at the time of filing of the subject application. Many strains that metabolize aromatic compounds, whether described in the art, or not, are optionally used as sources of suitable dioxygenase genes and enzymes for the present invention.

General methods for cloning and isolation of genes, e.g., arene dioxygenase genes encoding enzymes having aromatic ring dihydroxylating catalytic activity, are well known in the art. One of skill can isolate many new arene dioxygenase genes from microorganisms inhabiting soils, sediments, sewage treatment sludges, and aquatic environments. Enrichment cultures are useful for isolation of new strains with dioxygenase genes, particularly when the enrichment culture is established using an aromatic substrate and sample material comprising soil, water, sediments, and sludges from environments with substantial exposure to aromatic compounds, such as substituted benzenes.

Typically, isolation of new dioxygenase genes from any of the above microorganisms is guided by exemplifying approaches such as sequence homology, e.g., using hybridization probes comprising known genes, their fragments, or synthetic degenerate or non-degenerate oligonucleotides, with those dioxygenase genes that already display some degree of desired catalytic activity with aromatic substrates, including benzene and any other substituted benzenes.

Screening cloned libraries of unknown genes for ability to form readily detectable reaction products which are indicative of dioxygenase activity, e.g., alone or in a combination with enzymes effecting subsequent transformations of aromatic biodegradation pathways, is also optionally used to identify new dioxygenase genes useful in the present invention. Examples of such reactions are known in the art, and are exemplified by the formation of indigo from indole, whether substituted or not; by the formation of colored catechol meta-cleavage products from non-hydroxylated aromatic substrates, e.g., that are converted to these products via a sequence of associated activities of an arene dioxygenase, cis-dihydrodiol dehydrogenase and catechol dioxygenase (meta-cleaving); and by the formation of catechols from non-hydroxylated aromatic substrates by action of an arene dioxygenase (and arene cis-dihydrodiol dehydrogenase, where the diene diol product does not undergo spontaneous re-aromatization to catechol).

Arene dioxygenases with suitable catalytic activity towards p-xylene and substituted benzenes such as those represented by Formula (3) are optionally used in many different ways in the biocatalytic conversion step in the present invention. In one embodiments, wild type microbial isolates, having the desired dioxygenase activity, are subjected to different methods of mutagenesis known in the art (chemical, UV, transposons, etc) to obtain mutants lacking arene cis-diol dehydrogenase activity. Examples of known mutants in the art are P.putida F1/39D, P.putida RE213, and Pseudomonas sp. UV4. In addition to blocking activity of the arene cis-dihydrodiol dehydrogenase, other mutations are optionally introduced, including those allowing for constitutive expression of the dioxygenase. Use of any such mutants is well known to those of skill in the art and within the scope of the present invention.

However, to obtain improved performance in the biocatalytic step, e.g., in arene cis-diol-diene yield and rate of formation, suitable dioxygenase genes, e.g., arene dioxygenase genes, are cloned and expressed in a microbial host that naturally lacks arene cis-diol dehydrogenase activity, on a plasmid or other extrachromosomal expression vector and/or on a chromosome. Expression of dioxygenase genes is optionally achieved under a variety of promoters and expression control genes and proteins known in the art to allow display of sufficient arene dioxygenase activity. For one skilled in the art, it is possible to design many various arrangements of dioxygenase genes in the host microbial strain and to locate them on a chromosome and/or on one or more extrachromosomal replicons, e.g. plasmids. The latter is optionally the same or of different type and sequence. The sets of dioxygenase genes encoding subunits of the enzyme can be located on one replicon or distributed between several replicons. Additional copies of genes encoding individual subunits of arene dioxygenases are optionally incorporated into the host microorganisms. In the case of multiple copies of genes encoding dioxygenase polypeptides, the copies that encode functionally similar subunits optionally have the same sequence or variant sequences, as they are optionally recruited from different sources. In addition, they also optionally represent various mutants or chimeras derived from one or more ancestor gene(s).

Methods of Improving Dioxygenase Activity

Wild-type dioxygenases and mutants, chimeras, and variants as discussed above are all optionally used to enzymatically oxidize substituted benzenes, e.g., as a first step in preparing furanones. For example, a dioxygenase from Pseudomonas putida F1/39D is optionally used to enzymatically oxidize p-xylene and other substituted benzenes. However, improved dioxygenases are also desirable, e.g., to provide higher rates of formation for industrial applications. Methods of making polynucleotides encoding dioxygenases with desired catalytic activity are provided in U.S. Ser. No. 60/148,850, by Selifonov, and in PCT publication WO 01/12791 by Selifonov et al., published Feb. 22, 2001, both entitled, “DNA Shuffling of Dioxygenases for Production of Industrial Chemicals.”

A variety of recombination and recursive recombination (e.g., DNA shuffling) reactions and/or other diversity generating reactions, in addition to or concurrent with standard cloning methods, are optionally used to produce dioxygenases with desired properties. A variety of such reactions are known to those of skill in the art, including those developed by the inventors and their co-workers.

The following publications describe a variety of recursive recombination procedures and/or methods that can be incorporated into such procedures: Stemmer, et al., (1999) “Molecular breeding of viruses for targeting and other clinical properties. Tumor Targeting” 4:1-4; Nesset al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameriet al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proceedings of the National Academy of Sciences, U.S.A. 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proceedings of the National Academy of Sciences, U.S.A. 91:10747-10751.

Additional details regarding DNA shuffling methods are found in U.S. Patents by the inventors and their co-workers, including: U.S. Pat. No. 5,605,793 to Stemmer (Feb. 25, 1997), “METHODS FOR IN VITRO RECOMBINATION;” U.S. Pat. No. 5,811,238 to Stemmer et al. (Sep. 22, 1998) “METHODS FOR GENERATING POLYNUCLEOTIDES HAVING DESIRED CHARACTERISTICS BY ITERATIVE SELECTION AND RECOMBINATION;” U.S. Pat. No. 5,830,721 to Stemmer et al. (Nov. 3, 1998), “DNA MUTAGENESIS BY RANDOM FRAGMENTATION AND REASSEMBLY;” U.S. Pat. No. 5,834,252 to Stemmer, et al. (Nov. 10, 1998) “END-COMPLEMENTARY POLYMERASE REACTION,” and U.S. Pat. No. 5,837,458 to Minshull, et al. (Nov. 17, 1998), “METHODS AND COMPOSITIONS FOR CELLULAR AND METABOLIC ENGINEERING.”

In addition, details and formats for DNA shuffling are found in a variety of PCT and foreign patent application publications, including: Stemmer and Crameri, “DNA MUTAGENESIS BY RANDOM FRAGMENTATION AND REASSEMBLY” WO 95/22625; Stemmer and Lipschutz “END COMPLEMENTARY POLYMERASE CHAIN REACTION” WO 96/33207; Stemmer and Crameri “METHODS FOR GENERATING POLYNUCLEOTIDES HAVING DESIRED CHARACTERISTICS BY ITERATIVE SELECTION AND RECOMBINATION” WO 97/0078; Minshull and Stemmer, “METHODS AND COMPOSITIONS FOR CELLULAR AND METABOLIC ENGINEERING” WO 97/35966; Punnonen et al. “TARGETING OF GENETIC VACCINE VECTORS” WO 99/41402; Punnonen et al. “ANTIGEN LIBRARY IMMUNIZATION” WO 99/41383; Punnonen et al. “GENETIC VACCINE VECTOR ENGINEERING” WO 99/41369; Punnonen et al. OPTIMIZATION OF IMMUNOMODULATORY PROPERTIES OF GENETIC VACCINES WO 9941368; Stemmer and Crameri, “DNA MUTAGENESIS BY RANDOM FRAGMENTATION AND REASSEMBLY” EP 0934999; Stemmer “EVOLVING CELLULAR DNA UPTAKE BY RECURSIVE SEQUENCE RECOMBINATION” EP 0932670; Stemmer et al., “MODIFICATION OF VIRUS TROPISM AND HOST RANGE BY VIRAL GENOME SHUFFLING” WO 9923107; Apt et al., “HUMAN PAPILLOMA VIRUS VECTORS” WO 9921979; Del Cardayre et al. “EVOLUTION OF WHOLE CELLS AND ORGANISMS BY RECURSIVE SEQUENCE RECOMBINATION” WO 9831837; Patten and Stemmer, “METHODS AND COMPOSITIONS FOR POLYPEPTIDE ENGINEERING” WO 9827230; Stemmer et al., and “METHODS FOR OPTIMIZATION OF GENE THERAPY BY RECURSIVE SEQUENCE SHUFFLING AND SELECTION” W09813487.

Certain U.S. Applications provide additional details regarding DNA shuffling and related techniques, including “SHUFFLING OF CODON ALTERED GENES” by Patten et al. filed Sep. 29, 1998, (U.S. Ser. No. 60/102,362), Jan. 29, 1999 (U.S. Ser. No. 60/117,729), and Sep. 28, 1999, U.S. Ser. No. 09/22588 (Attorney Docket Number 20-28520US/PCT); “EVOLUTION OF WHOLE CELLS AND ORGANISMS BY RECURSIVE SEQUENCE RECOMBINATION”, by del Cardyre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Feb. 5, 1999 (U.S. Ser. No. 60/118,813) and filed Jun. 24, 1999 (U.S. Ser. No. 60/141,049) and filed Sep. 28, 1999 (U.S. Ser. No. 09/408,392, Attorney Docket Number 02-29620US); and “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393, Attorney Docket Number 02-010070US); and “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118854, U.S. Ser. No. 09/416,375 and U.S. Ser. No. 09/494,282).

As review of the foregoing publications, patents, published applications and U.S. patent applications reveals, shuffling (or “recursive recombination”) of nucleic acids to provide new nucleic acids with desired properties is optionally carried out by a number of established methods. Any of these methods can be adapted to the present invention to evolve the dioxygenases, e.g., arene dioxygenases, discussed herein to produce new dioxygenases with improved properties. Both the methods of making such dioxygenases and the dioxygenases produced by these methods are a feature of the invention.

In brief, at least five different general classes of recombination methods are applicable to the present invention. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo, e.g., by allowing recombination to occur between nucleic acids in cells. Third, whole cell genome recombination methods can be used in which whole genomes of cells are recombined, optionally including spiking of the genomic recombination mixtures with desired library components such as dioxygenase nucleic acids. Fourth, synthetic recombination methods are optionally used, in which oligonucleotides corresponding to different dioxygenases are synthesized and reassembled in PCR or ligation reactions which include oligonucleotides which correspond to more than one parental nucleic acid, thereby generating new recombined nucleic acids. Oligonucleotides can be made by standard nucleotide addition methods, or by tri-nucleotide synthetic approaches. Fifth, in silico methods of recombination can be effected in which genetic algorithms are used in a computer to recombine sequence strings which correspond to dioxygenases such as those listed in Table 1. The resulting recombined sequence strings are optionally converted into nucleic acids by synthesis of nucleic acids that correspond to the recombined sequences, e.g., in concert with oligonucleotide synthesis/gene reassembly techniques. Any of the preceding general recombination formats is optionally practiced in a reiterative fashion to generate a more diverse set of recombinant nucleic acids.

The above references provide these and other basic recombination formats as well as many modifications of these formats. Regardless of the format that is used, the nucleic acids of the invention are optionally recombined (with each other or with related (or even unrelated) nucleic acids) to produce a diverse set of recombinant nucleic acids, including homologous nucleic acids. In general, the sequence recombination techniques described herein provide particular advantages in that they provide for recombination between the nucleic acids of Table 1 or derivatives thereof, in any available format, thereby providing a very fast way of exploring the manner in which different combinations of sequences can affect a desired result. For example, desired results for improved dioxygenases include, but are not limited to, the ability to oxidize a different substrate, e.g., benzenes comprising a variety of substituents, or improved ability to oxidize an established substrate.

DNA shuffling and related techniques provide a robust, widely applicable, means of generating diversity useful for the engineering of proteins, pathways, cells and organisms with improved characteristics. In addition to the basic formats described above, it is sometimes desirable to combine recombination methodologies with other techniques for generating diversity. In conjunction with (or separately from) recombination-based methods, a variety of diversity generation methods can be practiced and the results (i.e., diverse populations of nucleic acids) evaluated. Additional diversity can be introduced into nucleic acids by methods that result in the alteration of individual nucleotides or groups of contiguous or non-contiguous nucleotides, e.g., mutagenesis methods. Mutagenesis methods include, for example, recombination (PCT/US98/05223; Publ. No. WO98/42727); oligonucleotide-directed mutagenesis (for review see, Smith, Ann. Rev.Genet. 19: 423-462 (1985)); Botstein and Shortle, Science 229: 1193-1201 (1985); Carter, Biochem. J. 237: 1-7 (1986); Kunkel, “The efficiency of oligonucleotide directed mutagenesis” in Nucleic acids & Molecular Biology, Eckstein and Lilley, eds., Springer Verlag, Berlin (1987)). Included among these methods are oligonucleotide-directed mutagenesis (Zoller and Smith, Nucl. Acids Res. 10: 6487-6500 (1982), Methods in Enzymol. 100: 468-500 (1983), and Methods in Enzymol. 154: 329-350 (1987)) phosphothioate-modified DNA mutagenesis (Taylor et al., Nucl. Acids Res. 13: 8749-8764 (1985); Taylor et al., Nucl. Acids Res. 13: 8765-8787 (1985); Nakamaye and Eckstein, Nucl. Acids Res. 14: 9679-9698 (1986); Sayers et al., Nucl. Acids Res. 16:791-802 (1988); Sayers et al., Nucl. Acids Res. 16: 803-814 (1988)), mutagenesis using uracil-containing templates (Kunkel, Proc. Nat'l. Acad. Sci. USA 82: 488-492 (1985) and Kunkel et al., Methods in Enzymol. 154:367-382)); mutagenesis using gapped duplex DNA (Kramer et al., Nucl. Acids Res. 12: 9441-9456 (1984); Kramer and Fritz, Methods in Enzymol. 154:350-367 (1987); Kramer et al., Nucl. Acids Res. 16: 7207 (1988)); and Fritz et al., Nucl. Acids Res. 16: 6987-6999 (1988)). Additional suitable methods include point mismatch repair (Kramer et al., Cell 38: 879-887 (1984)), mutagenesis using repair-deficient host strains (Carter et al., Nucl. Acids Res. 13: 4431-4443 (1985); Carter, Methods in Enzymol. 154: 382-403 (1987)), deletion mutagenesis (Eghtedarzadeh and Henikoff, Nucl. Acids Res. 14: 5115 (1986)), restriction-selection and restriction-purification (Wells et al., Phil. Trans. R. Soc. Lond. A 317: 415-423 (1986)), mutagenesis by total gene synthesis (Nambiar et al., Science 223: 1299-1301 (1984); Sakamar and Khorana, Nucl. Acids Res. 14: 6361-6372 (1988); Wells et al., Gene 34:315-323 (1985); and Grundstrom et al., Nucl. Acids Res. 13: 3305-3316 (1985). Kits for mutagenesis are commercially available (e.g., Bio-Rad, Amersham International, Anglian Biotechnology).

Other relevant references which describe methods of diversifying nucleic acids include Schellenberger U.S. Pat. No. 5,756,316; U.S. Pat. No. 5,965,408; Ostermeier et al. (1999) “A combinatorial approach to hybrid enzymes independent of DNA homology” Nature Biotech 17:1205; U.S. Pat. No. 5,783,431; U.S. Patent No.5,824,485; U.S. Pat. 5,958,672; Jirholt et al. (1998) “Exploiting sequence space: shuffling in vivo formed complementarity determining regions into a master framework” Gene 215: 471; U.S. Pat. No. 5,939,250; WO 99/10539; WO 98/58085 and WO 99/10539.

Any of these or other available diversity generating methods can be combined, in any combination selected by the user, to produce nucleic acid diversity, which may be screened for using any available screening method.

In the context of the present invention, screening can include testing for and identifying dioxygenase activities, by any of the assays in the art. In addition, useful properties such as the ability to oxidize a variety of substrates can also be selected for. A variety of dioxygenase related (or even unrelated) properties are optionally assayed for, using any available assay.

A recombinant nucleic acid produced by recursively recombining one or more polynucleotides of the invention with one or more additional nucleic acid also forms a part of the invention. The one or more additional nucleic acid may include another polynucleotide of the invention; optionally, alternatively, or in addition, the one or more additional nucleic acid can include, e.g., a nucleic acid encoding a naturally-occurring dioxygenase or a subsequence thereof, any homologous dioxygenase sequence or subsequence thereof, or any dioxygenase sequence as found in GenBank or other available literature, or, e.g., any other homologous or non-homologous nucleic acid (certain recombination formats noted above, notably those performed synthetically or in silico, do not require homology for recombination).

The recombining steps may be performed in vivo, in vitro, or in silico as described in more detail in the references above. Also included in the invention is a cell containing any resulting recombinant nucleic acid, nucleic acid libraries produced by recursive recombination of the nucleic acids set forth herein, and populations of cells, vectors, viruses, plasmids, or the like comprising the library or comprising any recombinant nucleic acid resulting from recombination (or recursive recombination) of a nucleic acid as set forth herein with another such nucleic acid, or an additional nucleic acid. Corresponding sequence strings in a database present in a computer system or computer readable medium are also a feature of the invention.

The above methods are optionally used in the present invention to provide improved dioxygenases, e.g., dioxygenases having greater oxidizing activity in the sense of higher conversion rates, e.g., conversion of substituted benzene to diol-diene compound, and/to greater or broader substrate specificity. For example, improved dioxygenases of the invention optionally convert p-xylene to 1,2-dihydroxy-3,6-dimethylhexa-3,5-diene faster than a wild-type dioxygenase or with a better conversion rate, e.g., a greater percentage of the p-xylene is converted. Alternatively, improved dioxygenases are useful for substrates that are not converted by wild-type dioxygenases, e.g., various substituted benzenes and other arene compounds.

The above diversity-generating methods are used in the present invention to provide improved dioxygenases, e.g., by shuffling. For example, DNA fragments encoding parental enzymes, e.g., wild-type dioxygenases such as those listed above and in Table 1, are recombined to produce a library of recombinant DNA segments. Typically, at least one of the parental enzymes encodes a dioxygenase that oxidizes a substituted benzene. The recombination steps are optionally repeated to produce more recombinant libraries, which are screened to identify DNA segments that encode dioxygenases with improved or enhanced activity, e.g., greater oxidizing activity than the parental enzymes. Multiple rounds of recombinations are optionally performed to provide even greater oxidizing activity.

Screening for improved dioxygenase activity is described, e.g., in U.S. Ser. No. 60/148,850, by Selifonov, entitled, “DNA Shuffling of Dioxygenases for Production of Industrial Chemicals.” Typically, screening comprises introducing a library of recombinant polynucleotides into a population of microorganisms and placing the microorganisms in a medium comprising a substrate of interest, e.g., a substituted benzene from which a desired furanone can be made using the methods of the present invention. Those organisms exhibiting improved activity toward the substrate, e.g., as compared to a parental or wild-type enzyme, are identified. The improved activity typically comprises greater oxidation activity or activity toward a substrate not typically oxidized by the parental or wild-type enzyme.

The improved activity is typically monitored using one or more techniques such as thin layer chromatography, high performance liquid chromatography (HPLC), chiral HPLC, mass-spectrometry, NMR spectroscopy, radioactivity detection from a radioactively labeled compound, e.g., labeled diols, scintillation proximity assays, or UV spectroscopy. These techniques are well known to those of skill in the art and are described in detail in U.S. Ser. No. 60/148,850, by Selifonov, entitled, “DNA Shuffling of Dioxygenases for Production of Industrial Chemicals.”

The improved enzymes produced are then optionally used to oxidize substituted benzenes, as described above, as a first step in the preparation of 4-hydroxy-2,5-dimethyl-2,3-dihydrofuran-3-one and other furanones.

The invention also includes compositions comprising two or more dioxygenases of the invention (e.g., as substrates for recombination). The composition can comprise a library of recombinant nucleic acids, where the library contains at least 2, 3, 5, 10, 20, or 50 or more nucleic acid species. The nucleic acids are optionally cloned into expression vectors, providing expression libraries, which are also an aspect of the invention.

Other variations involving host microorganisms are also available for improving biocatalysis of substituted benzenes to diol-diene compounds. For example, host strains are optionally used that exhibit increased levels of cell resistance to large concentrations of aromatic products and their desired oxidized products. Host organisms that naturally possess high aromatic solvent resistance are optionally used. See, e.g., U.S. Ser. No. 60/148,850 and references therein. Alternatively, novel microbial strains having the ability to tolerate large concentrations of the compounds of interest, e.g., substituted benzenes, are readily isolated by one of skill in the art, e.g., using enrichment cultures, e.g., from soil, sediment, sludge, and water samples in the presence of substituted benzenes, such as p-xylene or other compounds having similar structures and/or physical properties. These cultures are optionally performed with or without the addition of carbon sources. Typically, such cultures are set in the presence of additional carbon sources to isolate strains that tolerate supersaturating concentrations of p-xylene and other substituted benzenes but do not utilize these compounds as a carbon source. One of skill in the art can easily introduce and practice many variations regarding host organism properties and selection of these properties.

Methods and Conditions for Enzymatic Oxidation of Aromatic Substrates

To enzymatically oxidize aromatic substrates, the substrate, e.g., a substituted benzene such as p-xylene, is contacted, e.g., in the presence of water and/or an organic solvent, with a dioxygenase, e.g., toluene dioxygenase or any other dioxygenase described above. Alternatively, the substrate is contacted with one or more cells that possess dioxygenase activity, e.g., the cells express a dioxygenase that oxidizes the aromatic substrate of interest. For example, a cell, e.g., a microbial or bacterial cell, with dioxygenase activity in the present invention expresses an enzyme that is capable of dihydroxylating p-xylene to form cis-1,2-dihydroxy-3,6-dimethylhexa-3,5-diene. In addition, the enzyme typically oxidizes other substituted benzenes as represented by Formula (3) to form compounds having Formula (5).

Small-scale oxidations are optionally carried out in flasks, e.g., with air-permeable closures, whereby aeration and stirring is provided by shaking. Such methods are well known in the art. Preferred conditions for oxidation of p-xylene and other substituted benzenes include carrying out of the oxidation reaction under aerobic conditions, e.g., in a fermentor in which oxygen is provided by passing air through an aqueous liquid media stirred by means of agitation/impellers.

Aromatic substrates such as p-xylene and substituted benzenes typically have a low aqueous solubility and a high volatility. After sufficient cell density has been reached in the fermentor, and conditions for expressing the arene dioxygenase have been achieved, the aromatic substrates are optionally administered in a variety of ways. For example, passing air saturated with the substrate vapor through the fermentor, portionwise small additions of the substrate directly to the medium, or controlled-rate small additions directly to the medium are optionally used to introduce substrate into the medium where it is oxidized by the expressed dioxygenase. The rate of addition is typically controlled in such a way that substrate concentration does not exceed limits of the toxicity to the host cells, and so that the rate of addition does not substantially exceed rate of bio-oxidation. This keeps losses of volatile substrates with air flow at a minimum.

If a solvent-resistant microbial host is used, the water-immiscible substrate is optionally added in excess to form a second phase, in a neat form, or in a mixture with inert non-metabolizable solvent. Oxidation products, e.g., compounds having Formula (4) or (5) typically accumulate in the aqueous medium, however, if biphasic systems and solvent-resistant host strains are used, the desired products can partition to the organic phase, thus facilitating product recovery and providing conditions for continuous product removal from the aqueous phase.

Typically, during the oxidation reaction, a sufficient amount of a utilizable carbon source is present in the medium so that the reducing cofactors used in arene dioxygenase activity are regenerated within the cells. The oxidation reaction in the fermentor is typically carried out until desired levels of diol-diene product have been reached or until oxidation no longer takes place due to decrease in arene dioxygenase activity. The diol-diene compounds are typically recovered from the reaction medium and used in further steps in the preparations of 4-hydroxy-3[2H]-furanones.

Recovery of Diol-dienes from the Reaction Medium

Compounds having Formula (4) and/or (5) are produced according to the enzymatic oxidation methods described above, e.g., by contacting a substituted benzene with a dioxygenase. The benzenes are oxidized to form diol-diene compounds. The typical method involves growing cells that express one or more enzyme having arene dioxygenase activity. The substrate is added to the cells, where it is oxidized. The diol-diene compounds are typically isolated from the cell medium as described below.

Typically the microbial cells used for the above-described biocatalysis are removed from the medium by means known in the art, such as centrifugation, lysis, flocculation, or membrane filtration. Active cells removed by centrifugation or filtration are optionally reused for re-inoculation of a biocatalysis medium.

Recovery of diol-diene compounds, e.g., arene cis-dihydrodiols, from an aqueous biocatalysis medium is readily achieved by liquid-liquid extraction using a variety of organic water-immiscible solvents, such as low-boiling esters, ethers, alcohols, ketones, aromatic hydrocarbons, terpenoids, halogenated solvents, and the like. It is apparent to one s killed in the art that these are the non-limiting examples of solvents and other solvents are optionally used, either individually or in mixtures, to provide for satisfactory isolation of the diol-diene compounds. For example, ethyl acetate is optionally used to extract diol-dienes, such as cis-1,2-dihydroxy-3,6-dimethylhexa-3,5-diene, from the culture medium.

Extractions are optionally performed in batches, or continuously using various flow-through extractors known in the art. Various relative ratios of aqueous medium and solvents are optionally used as well as repeated extractions with the same or different solvents. Compounds that increase ionic strength of the aqueous medium, e.g., inorganic salts such as NaCl, as well as those that improve liquid-liquid phase separation are optionally added. Various solvents and methods of extractions are known by and optionally used by those of skill in the art to extract the diol-diene products and, e.g., to improve extraction efficiency and decrease the overall cost of extraction step.

Alternatively, the aqueous medium from the biocatalytic step is concentrated or evaporated to dryness, e.g., under reduced pressure. Different solid-phase extraction techniques, as well as precipitation of the arene-cis-diols by arylboronic or alkylboronic acids known in the art are also optionally applied for recovery of the diol-diene compounds produced by enzymatic oxidation of substituted benzenes.

Typically, the pH of the aqueous medium during extraction procedures is maintained in the range from about 4 to about 9, more typically in the range between about 5.5 to about 8, e.g., to avoid acid- or base-catalyzed dehydration of the arene cis-dihydrodiols to the corresponding phenols. The temperature of the aqueous medium, extraction mixture, and the solvent extracts is typically in the range between about −5 to about 60° C., more typically between about 0 and about 45° C., e.g., to avoid heat-induced dehydration of the diol-dienes to corresponding phenols. Essentially pure crystalline arene diol-dienes are thus obtained by removing the extraction solvent under reduced pressure to dryness. The extracted arene diol-dienes are optionally used immediately for subsequent procedures, or stored, typically in a freezer below 0° C., e.g., in solid/crystalline form or in solutions in suitable solvents. Before storage, traces of acids are optionally removed from batches of the extracted arene diol-dienes, e.g., if they are to be stored for a prolonged time.

The diol-diene compounds produced from enzymatic oxidation typically comprise compounds having Formulas (4) and (5). Some compounds, e.g., those of Formula (4) and those of Formula (5) in which R₅ and R₆ are the same, are symmetrical achiral diol-dienes. In other embodiments, e.g., when R₅ and R₆ of Formula (5) are different, the diol-diene is a chiral molecule. In addition, the enzymatic oxidation produces substantially all cis-stereoisomers. These molecules are used in the following chemical synthesis steps to produce the furanones described above. The diol-diene compounds produced and recovered as described above are optionally used to chemically synthesize furanones, e.g., 4-hydroxy-2,5-dimethyl-2,3-dihydrofuran-3-one.

III. Chemical Oxidation of Diol-diene Compounds to Form Diol-dione Compounds

The diol-diene compounds produced as described above, e.g., by enzymatic oxidation, are typically chemically oxidized to form diol-dione compounds as shown in Formulas (6) and (7), e.g., hexane-3,4-cis-diol-2,5-dione. The hexene ring is typically broken to form two ketone groups. The oxidation reaction typically comprises contacting the diol-diene with one or more oxidizing reagent, e.g., alkali metal salts, alkali metal permanganate salts, alkali metal periodate salts, alkali metal hypochlorite salts, organic peroxyacids, organic peroxides, inorganic peroxyacids, inorganic peroxides, ozone, and the like. “Oxidizing reagent” is used herein to refer to compounds that are typically mixed with a diol-diene compound as described above to convert it to a dione compound. Such reagents typically bring about an increase in oxidation state of the diol-diene compound, e.g., concurrent with a reduction in one or more atoms of the oxidizing reagent. Catalytic amounts of ruthenium halide or oxide are also optionally used to oxidize the diol-diene compounds of the present invention into diol-dione compounds.

In one embodiment, the diol groups of the diol-diene compounds are protected before the chemical oxidation and the resulting protected-dione compound is deprotected, e.g., to be used in further steps. Alternatively, an unprotected diol-diene compound is oxidized using ozonolysis, e.g., in the presence of a boric acid derivative. When chemical oxidation is performed on protected diol-diene compounds, the resulting compounds comprise protected dione compounds, e.g., compounds having Formula (12), (13), (14), and (15). When chemical oxidation is performed on unprotected compounds, the resulting compound is a diol-dione, e.g., a compound having Formula (6) or (7).

Protection of the Diol-diene Hydroxyl Groups Prior to Oxidation

For the purpose of this invention, various known and common protection reactions and reagents are optionally used to protect the diol groups in diol-diene compounds from oxidation. The diol-diene compounds produced by enzymatic oxidation are oxidized, e.g., chemically, to form diol-dione compounds. To prevent the diol groups from being oxidized during this step, protecting groups are optionally used. Such protection groups include, but are not limited to, formation of esters, e.g., esters of carboxylic and boronic acids, ethers, e.g., ethers of tertiary alcohols, silyl ethers, cyclic ketals, and cyclic acetals. Several alternative methods and conditions for the protection of diol dienes have been reviewed, e.g., by Brown and Hudlicky, Organic Synthesis: Theory and Applications 2, 113-176 (1993).

In one embodiment, cyclic ketals and cyclic acetals are used to protect the diol-diene compounds produced by enzymatic oxidation, e.g., those derived from p-xylene and the other substituted benzenes. The formation of cyclic ketals and acetals is typically accomplished by reaction of a diol-diene having Formula (4) or (5) with about a 2 to about a 100-fold excess of low boiling ketones, aldehydes, ketals, acetals, or mixtures of ketone and ketal, or aldehyde and acetal. Catalytic amounts of mineral or organic acids are optionally used to facilitate the reaction, and additional suitable solvents, such as hydrocarbons, aromatic hydrocarbons, ethers, esters and halogenated solvents are optionally used for the diol-diene-acetal or diol-diene-ketal formation. For example, aryl or alkylsulfonic acid are optionally used to catalyze the formation of a cyclic ketal or cyclic acetal on a diol-diene. Other useful catalysts include, but are not limited to, solid phase catalysts, e.g., solid phase acids, and resins comprising protonated sulfonic groups.

The protection reaction is typically stopped by neutralizing the catalyst, e.g., an acid catalyst or solid phase catalyst. Neutralization of the catalyst is typically carried out by addition of a suitable acid-scavenging reagent, e.g., sodium bicarbonate, or by washing the organic solution with alkaline (pH about 7.5-10) aqueous solution of alkali metal carbonates, alkali, or alkaline buffers.

In one embodiment, protection reactions and reagents that allow for the formation of acetonides (cyclic ketals of acetone) are used. Such acetonides are optionally obtained by reacting a diol-diene with an excess of one or more of: 2,2-dimethoxypropane, 2,2-diethoxypropane, 2,2-dimethyl-1,3-dioxolane, 2-methoxypropene, 2-ethoxypropene, and acetone. As is apparent to one skilled in the art, these reagents can be used along with other co-solvents compatible with the reaction conditions. A preferred embodiment for the protection of diol-dienes is the use of an excess of acetone or acetone mixed with small amounts of 2,2-dimethoxypropane or 2,2-diethoxypropane. The preferred molar ratio of acetone to the 2,2-dimethoxypropane or 2,2-diethoxypropane is in the range of about 50:1 to about 2:1.

Various acid catalysts are optionally used for diol-diene-acetonide formation. Non-limiting examples of such acids include, but are not limited to, hydrochloric, sulfuric, camphorosulfonic, methanesulonic, triflic, benzenesulfonic, p-toluenesulfonic acids, as well as strong cation-exchange solid resins or gels known in the art, particularly those having sufficient number of equivalents of sulfonic acid groups in the protonated form. Preferred examples of acid catalysts of use in this invention are: p-toluenesulfonic acid and solid resins with sulfonic groups in the protonated form.

Prior to use, solid phase resins comprising protonated sulfonic groups, after equilibration to H⁺ form are preferably conditioned with acetone, or a with mixture of acetone and acetone ketal, e.g., to substantially remove water and/or other protic solvents from the resin matrix. After allowing sufficient time for formation of acetonide, typically with stirring at a temperature in the range between about 0° C. and about 40 ° C., the catalyst resin is removed by filtration. Alternatively, the solution of diol-diene in acetone, or in the mixture of acetone and acetone ketal, is passed one or more times through a column or reactor filled with a sufficient amount of the sulfonic acid catalyst resin.

The compounds, e.g., protected diol-diene compounds, formed upon protection of the hydroxyl groups of the diol-diene compounds include those having Formulas (8), (9), (10), and (11). In some embodiments, the protected diol-diene comprises a symmetrical achiral compound, e.g., when R₅ and R₆ in Formula (9) or (11) are the same. The protected compounds are then typically oxidized using a suitable oxidizing reagent to form protected dione compounds.

Oxidizing the Protected Compounds to Form Protected Dione Compounds

The diol-diene compounds or the protected diol-diene compounds are typically oxidized to form a diol-dione compound, e.g., those having Formula (6) or (7), or a protected dione compound, e.g., those compounds having Formula (12), (13), (14), or (15). The diol-diene compounds are typically oxidized by contacting them with an oxidizing reagent. Many reagents for alkene or diene oxidation and cleavage are known in the art; and such reagents, or their combinations, as well as varying oxidation reaction conditions, are optionally used to oxidize the protected diones of the present invention. Typically, the oxidizing reagents are selected from those known in the art that are compatible with the protection groups used to protect the vicinal hydroxyl groups of diol-diene compounds or from those reagents which do not oxidize the vicinal cis-diol moiety.

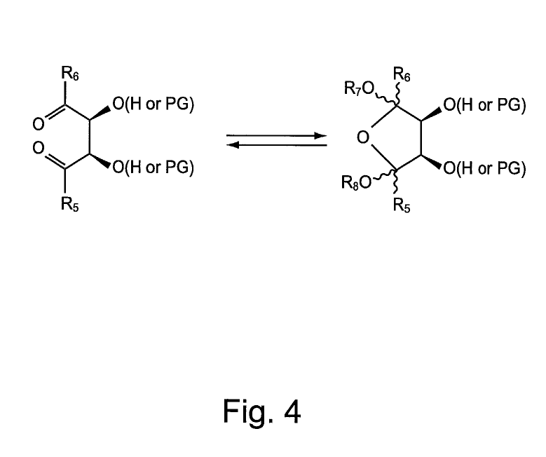

The dione compounds produced upon oxidation typically exist as free diones or as cyclic pseudofuranose ketals, or as an equilibrium thereof as shown in FIG. 4, in which where R₅, R₆ and the protecting groups (PG) are the same as defined above, and where R₇ and R₈ are each independently selected from hydrogen, alkyl, acyl or aralkyl.

Factors such as the nature of the solvent, e.g., water, alcohols, carboxylic acids, or lack thereof, temperature, chemical nature of protection groups, and the like can influence the shift in equilibrium between diones and ketals. Although the dione forms are shown throughout the present invention for the purpose of clarity of description, the ketals are recognized as synthetically equivalent compounds which one skilled in the art can use to accomplish preparation of furanones of interest, e.g., 4-hydroxy-3[2H]-furanones such as 4-hydroxy-2,5-dimethyl-2,3-dihydrofuran-3-one. Use of the cyclic pseudofuranose ketals for synthesis of furanone compounds is fully within the scope of the invention.

If protecting groups are used during the chemical oxidation step described above, the oxidized protected-dione compounds are typically deprotected prior to the next step, e.g., cyclization reaction described below, in the preparation of 4-hydroxy-3[2H]-furanones. If the oxidation from diene to dione was accomplished without the use of protecting groups, the resulting compounds are typically directly cyclized to form 4-hydroxy-3[2H]-furanones.

Permanganate and Periodate Oxidations

Oxidants compatible with acetonide, ester, or ether protection groups are known in the art and include, but are not limited to, alkali metal salts of permanganate e.g. NaMnO₄, KMnO₄, and the like, or a combination of permanganate with alkali metal salts of periodic acid, e.g., NaIO₄, KIO4, and the like. Sodium salts of permanganate and periodate are typically preferred. Reactions between the oxidizing reagent, e.g., and the ester or ether protected diene compounds are typically carried out with stirring, at a temperature between about −10° C. to about 30° C., and at a pH between about 6 and about 9. As is apparent to one skilled in the art, various solvents, including aqueous solutions and mixtures of water and water-miscible solvents, compatible with the above oxidants, are optionally used to effect oxidation and cleavage of protected dienes, e.g., compounds having Formula (8), (9), (10), or (11), or the like, to dione compounds, e.g., compounds having Formula (12), (13), (14), or (15), or the like. The amount of permanganate and periodate oxidants is typically calculated from the reaction stoichiometry such that at least about 10-20% molar excess of oxidant or oxidizing reagent is provided, in respect to the amounts of dienes in the cleavage reaction of both π-bonds of the diene to yield the diol-dione compounds of the invention. Typically the oxidants are pre-dissolved in water or another suitable solvent prior to mixing them with a solution of protected diol-dienes. The protected diones are then typically subjected to a deprotection reaction prior to cyclization to form 4-hydroxy-3[2H]-furanones.

Ozonolysis in Solution

In another embodiment, oxidation of dienes having Formula (4), (5), (8), (9), (10), or (11) to diones having Formula (6), (7), (12), (13), (14), or (15), is performed using ozonolysis. Protected and/or unprotected dienes are optionally oxidized by passing a gas stream with sufficient amounts of ozone or an ozone/oxygen mixture through a diol-diene solution. An ozone or ozone/oxygen mixture is readily and inexpensively generated by means of using commercially available ozonators. Solutions of diol-dienes, e.g., in organic solvents compatible with ozonolysis, in buffered water (pH between about 5 to about 8), or various mixtures thereof, are typically used to carry out oxidation by ozonolysis. When unprotected dienes, e.g., those having Formula (4) or (5) are used in ozonolysis, a sufficient amount, e.g., 1 equivalent or more, of free acids or of alkali metal salts of an acid, e.g., boric acid, alkylboronic acid, arylboronic acid, or the like, is optionally added to the solution of diol-dienes prior to ozone addition, e.g., to prevent oxidation of the hydroxy groups.

As known in the art, ozonolysis of dienes and alkenes yields highly labile ozonides which are typically decomposed to the corresponding keto compounds using one or more known procedures. Reductive decomposition of ozonides arising from ozonolysis of the diol-diene compounds of the invention, is optionally accomplished using one or more of a variety of common reagents, exemplified by sulfite inorganic salts, iodide inorganic salts, dimethylsulfide, and the like. Oxidative decomposition of the ozonides is optionally accomplished by addition of hydrogen peroxide or other suitable oxidizer, including alkali metal salts of peroxyacids, e.g., sodium percarbonate, sodium perborate, sodium or potassium persulfate, and the like. Hydrolytic decomposition of ozonides is typically accomplished by reacting ozonides with water, in the presence of catalytic amounts of a strong inorganic alkali or acid. These methods are well known and easily practiced by those skilled in the art to achieve the desired decomposition of ozonides to corresponding diones, e.g., diol-diones having Formula (6), (7), (12), (13), (14), or (15).

Ozonolysis of Diol-dienes on Boronate Resin

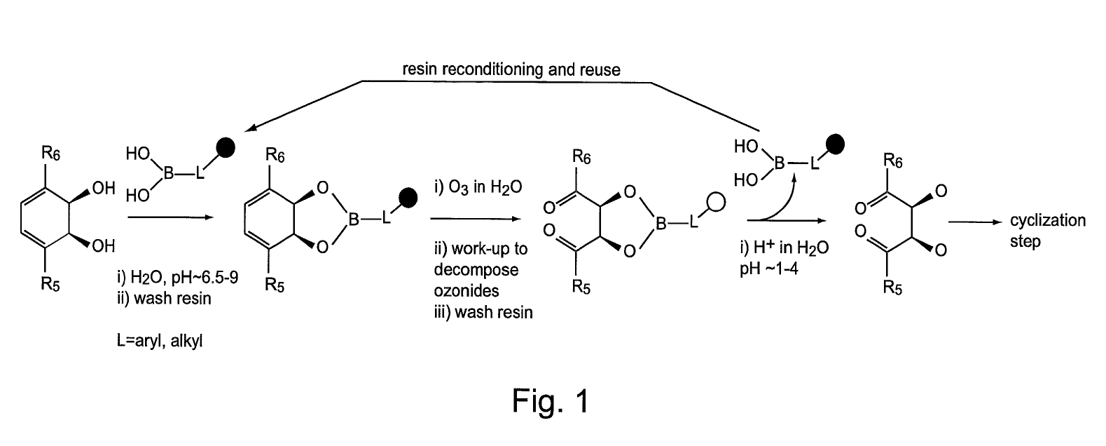

In another embodiment of the present invention, ozonolysis, to provide oxidation and cleavage of the diene moiety of the diol-diene compounds, is performed on a resin or one or more inorganic adsorbents. The resin or adsorbent typically contains a sufficient number of equivalents of alkylboronic or arylboronic moieties to oxidize the diol-dienes, and has a solid-phase or polymeric matrix that is chemically resistant to ozone, acids and water. A schematic of an oxidation reaction performed on a boronate resin is provided in FIG. 1 (R₅ and R₆ are the same as described above for Formulas (2), (3), (5), and the like).

Boronate-type resins are known in the art and can be prepared using well known chemistry. In this embodiment of the present invention, recovery of arene diol-dienes from a clarified aqueous biocatalysis medium is readily attained by passing the diol-diene through a column, or by adding it to a batch reactor containing the boronate resin, e.g., for large-scale industrial manufacturing of 4-hydroxy-2,5-dimethyl-2,3-dihydrofuran-3-one. Typically, other glycol compounds, such as soluble carbohydrates, glucose, or glycerol, are not present in the clarified aqueous biocatalysis medium to minimize the undesired competition between diol-dienes and the unrelated glycols for the boronate groups of the resin. In practice, such conditions are readily attained if glycols and carbohydrates are not used as carbon sources for microbial cells during the above-described biocatalysis step, or if the biocatalysis reaction is performed until the undesired glycol compounds are essentially completely utilized by the cells. During extraction of the diol-diene compounds by covalent attachment to the boronate resin, the pH of the aqueous medium is maintained in the range from about 6.5 to about 9 to promote the formation of cyclic boronate esters.

Prior to ozonolysis, the boronate resin loaded with diol-diene derivatives is typically conditioned with different suitable solvents, including fresh buffered aqueous solutions, pH typically about 7 to about 8.5, or suitable organic solvents that lack hydroxyl and carboxyl groups and are compatible with ozonolysis.

Such conditioned loaded resin is typically treated with ozone or an ozone/oxygen solution in a suitable solvent to complete diene cleavage to form a dione. The ozonides that are formed are typically decomposed by hydrolytic, e.g., neutral or alkaline, work-up, or by oxidative or reducing work-ups as described above. After decomposition of the ozonides, the boronate resin is optionally washed, e.g., with buffered water to remove any non-covalently bound products, and the desired diol dione compound is released from the medium by washing the resin, typically with acidified water, e.g., with a pH between about 1 and about 4. After adjustment of the pH to the slightly alkaline range, e.g., about 7.5 to about 9.5, the solution is optionally supplemented with catalysts and subjected to a cyclization reaction to form a furanone having Formula (1) or (2), e.g., a 4-hydroxy-3[2H]-furanone such as 4-hydroxy-2,5-dimethyl-2,3-dihydrofuran-3-one.

Other Oxidizing Reagents for Diol-diene Cleavage

It is evident to those skilled in the art that many other oxidizing reagents are optionally used to convert diol-diene compounds to diol-dione compounds. Examples of such reagents include, but are not limited to, ruthenium tetroxide used as a catalyst in the presence of another oxidant, e.g., in combination with hypochlorite inorganic salts, OsO₄/sodium periodate, alkaline hydrogen peroxide, or alkaline perborate, percarbonate, or persulfate.

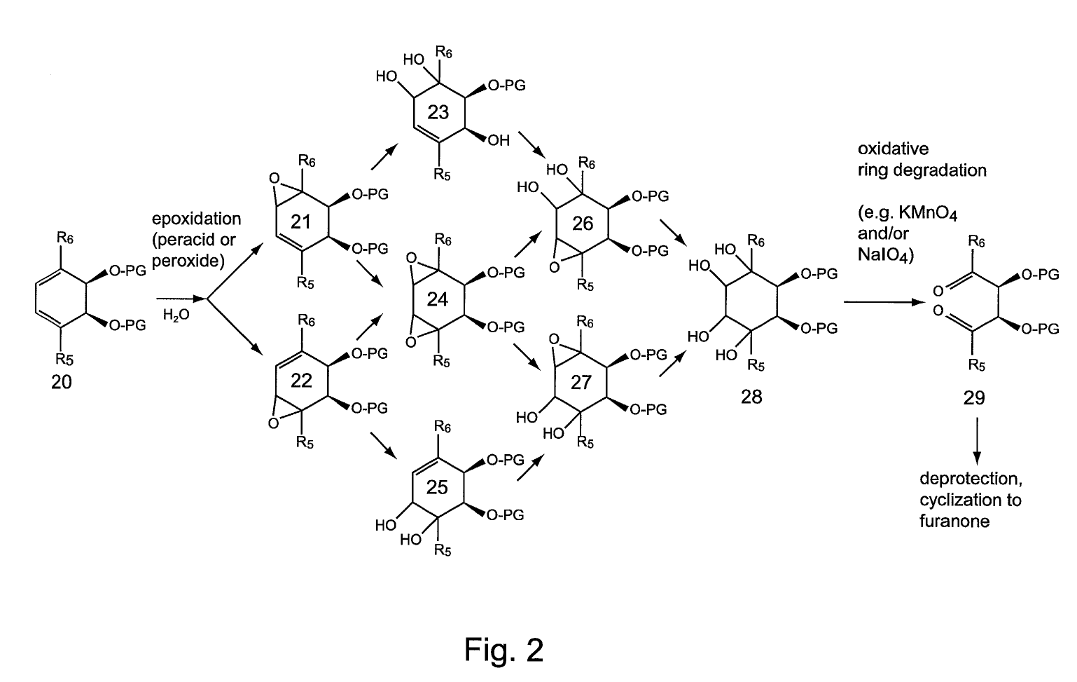

Alternative embodiments for effecting cleavage of dienes, e.g., compound 20 in FIG. 2, to diones, e.g., compound 29 in FIG. 2, are based on the use of epoxidizing reagents, e.g., 3-chloro-peroxybenzoate, o-peroxy-phthalate, peroxyacetate, peroxytrifluoroacetate, e.g., as free acids or salts, hydrogen peroxide, as well as by organic hydroperoxides such as t-butylperoxide. FIG. 2 provides an epoxidation reaction scheme for oxidation of diol-dienes to diol-diones (R₅ and R₆ are defined as described above and PG represents a protecting group such as those described above, e.g., R₁, R₂, R₃, R₄ in Formulas (8), (9), (10), and (11)). Epoxidizing reagents, as described above, when added to a protected diene, form epoxidized compounds, such as those of compounds 21 and 22. These compounds are optionally further converted, without isolation, e.g., in-situ from the reaction mixture, by action of suitable nucleophiles to produce cyclitol compounds, as shown by compound 28 in FIG. 2, via intermediates such as compounds 23-27 in the reaction scheme provided in FIG. 2. Water comprises a suitable nucleophile for this embodiment. Typically, the pH of the reaction mixture is compatible with the protection groups used.

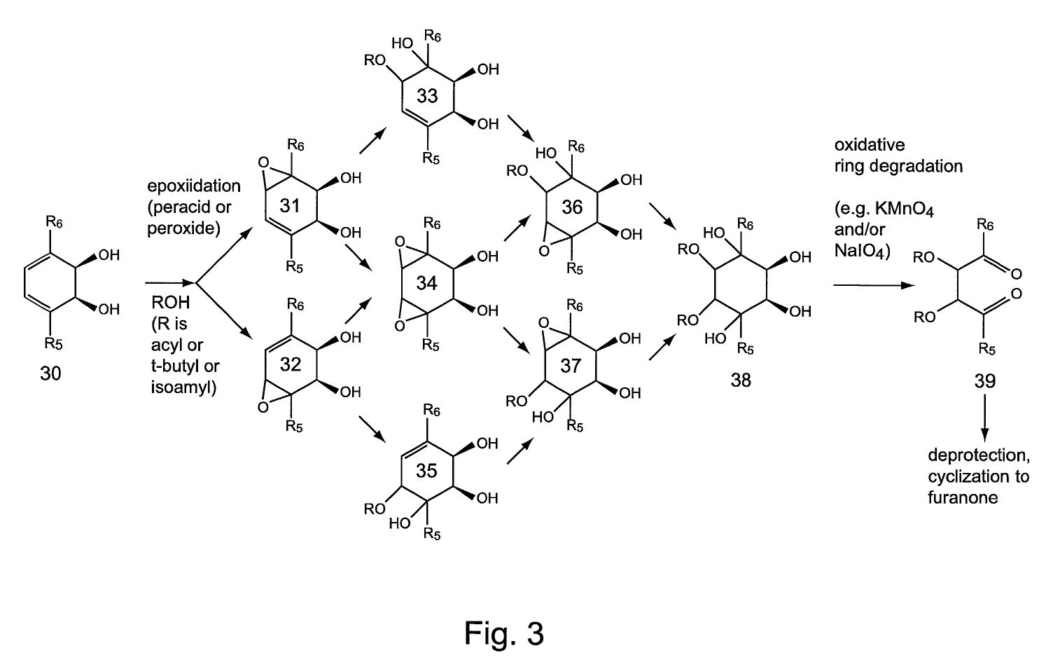

Similarly, unprotected dienes, such as compound 30 in FIG. 3, are converted in the presence of epoxidizing reagents and a suitable nucleophile to a cyclitol, e.g., compound 38, via intermediates 31-37. These conversions are illustrated in the reaction scheme shown in FIG. 3 (R₅ and R₆ are defined as above). Suitable nucleophiles for this embodiment include, but are not limited to, tert-butyl alcohol or other tertiary alcohols, benzyl alcohol, salts of carboxylic acids, and the like. The pH of the reaction mixture is typically maintained at initial stages of the reaction in a range between about 6 and about 8, e.g., to avoid dehydration of compound 30.

For further descriptions of oxidizing reagents, oxidation, epoxidation, and ozonolysis, see. e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York).

Deprotection of Protected Diol-dione Compounds

If the diol-diene compounds of the invention were protected before the chemical oxidation step described above, the next step in the preparation of 4-hydroxy-3[2H]-furanones is typically the deprotection of the protected diol-dione compounds. Depending on the nature of the protection groups used, different deprotection conditions and reagents are used to convert the protected diones, e.g., those having Formulas (12), (13), (14), or (15), to the desired dione diol compounds, e.g., compounds having Formula (6) or (7). Typically, a deprotecting reagent, e.g., ascetic acid, hydrochloric acid, sulfuric acid, phosphoric acid, or the like, is used to contact the protected compound. The deprotecting reagent aids in the removal of the protecting group from the dione compound and restoration of the diol groups to form unprotected diol-dione compounds.

If carboxylic esters have been used, then alkaline hydrolysis, or enzymatic hydrolysis, e.g., using one or more lipase or esterase enzyme known in the art is used. If boronic acid esters have been used, acidic hydrolysis is optionally used.

If acetonide, a cyclic isopropylidene derivative, or other cyclic ketal or cyclic acetal protection groups have been used to protect the hydroxyl groups of the diol-diene before chemical oxidation to the diol-dione, the protection group is typically removed by acid-catalyzed hydrolysis in water, or in another suitable solvent, or a mixture thereof. Acids suitable for removing this type of protection group include, but are not limited to, acetic, hydrochloric, sulfuric, phosphoric, oxalic, citric acids, or mixtures thereof. The amount of acid used typically maintains the pH of the reaction mixture in a range between about 0 and about 3. Water or a mixture of water and ethanol is a preferred embodiment for effecting the deprotection of protected diol-dione compounds to diol-dione compounds. In this case the resulting solution is optionally used directly in the next step, e.g., a cyclization reaction to provide the desired furanone, e.g., 4-hydroxy-2,5-dimethyl-2,3-dihydrofuran-3-one, or related furanones.

IV. Cyclization of Diol-dione Compounds to Make Furanones

The substituted benzenes of the present invention are biocatalytically oxidized to form diol-diene compounds that are typically chemically oxidized to form diol-dione compounds, e.g., compounds having Formulas (6) or (7). These compounds are easily cyclized into desired furanone compounds, e.g., compounds having Formula (1) or (2).

Methods to effect this cyclization reaction are known to those of skill in the art. See, e.g., U.S. Pat. No. 3,694,466 by Buchi et al., which provides methods of making 2,5-dimethyl-4,5-dihydrofuran-3-ol-4-one and recovery of the cyclized furanone product. These methods are also suitable for cyclization and recovery of furanones in the present invention. Alternatively, after cyclization, a solvent, such as propylene-1,2-glycol is added to the reaction mixture, and the products are distilled off in a manner similar to that described in U.S. Pat. No. 5,148,840 by Decnop et al.

In another embodiment deprotection and cyclization reactions are combined in a one-pot process. The pH of an aqueous solution comprising a compound of Formula (6) or Formula (7) resulting from removal of acetonide or a tert-butoxy-protection group, e.g., using dibasic or tribasic acids such as sulfuric, phosphoric, oxalic, or citric, is adjusted to have a pH in the range between about 6 and about 9. The adjustment is typically performed by addition of a suitable amount of alkali, or alkali metal carbonate. The reaction solution is brought to reflux conditions for sufficient time to effect the cyclization reaction, resulting in furanones of Formulas (1) and/or (2).

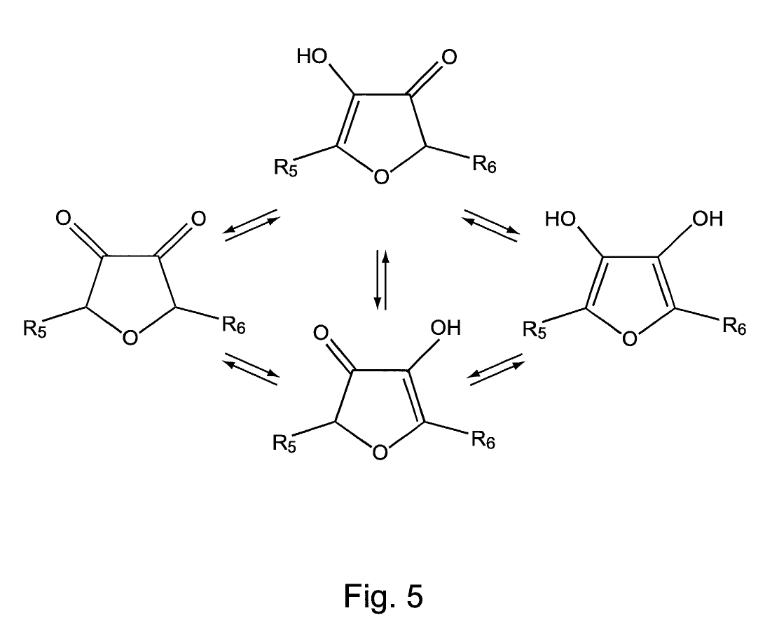

The furanones produced by the cyclization reaction, e.g., compounds having Formula (1) and/or (2) may exist in the form of tautomers, depending on the nature of the R₅ and R₆ substituents, solvent or lack thereof, pH, and temperature. The tautomeric forms of Formula (2) are provided in FIG. 5, and are considered the equivalent of compounds having Formula (2).

The entire process of making 4-hydroxy-3[2H]-furanones, as described above, is optionally carried out using a mixture of aromatic substrates as starting material. The mixture optionally includes any combination of p-xylene, substituted benzenes of Formula (3), and other aromatic compounds that are oxidized by the arene dioxygenases or improved arene dioxygenases as discussed above used to effect the biocatalytic oxidation step of the process. In this case, the process results in mixtures of various furanone products with different relative abundance of individual furanone compounds. Such mixtures are optionally used in preparing novel artificial flavor compositions. Typically, when selecting mixtures of aromatic substrates, compounds that are converted by arene dioxygenases to unwanted products, e.g., products other than diol-dienes, are avoided to lessen the chance of impurities and by-products being generated in the subsequent chemical steps.

### V. EXAMPLES

### Example 1

### Biocatalytic Oxidation of p-xylene

In an aerated agitated fermentor, 1.5 L of autoclaved BSM medium pH 7.0, 40 μM ferrous ammonium sulfate, 10 ml of 10% solution casamino acids in water, and 10 ml of 10% yeast extract solution in water were added. The temperature of the medium was brought to 37° C.

Ampicillin was added by means of a concentrated stock solution, to a final concentration of 100 μg/mL and glucose was added to a final concentration of 40 mM. The fermentor was inoculated with 100 ml of overnight culture of strain E.coli JM109 (Genbank number J04996) from Stratagene, (La Jolla, Calif.) (pDTG601a) (Zylstra and Gibson, 1991, supra) and grown in a shake flask in Luria-Bertrani medium with 100 μg/mL of ampicillin, to give a starting OD₆₀₀ of 0.165 in the fermentor. The air was supplied at 2.2 L per min, and the culture was grown until OD₆₀₀ 1.03 has been reached (approximately 3 hours). After that, the temperature was reduced to 30° C., another 40 mM of glucose, 10 ml of the 10% solution of casamino acids, 10 ml of the 10% yeast extract solution and 25 μg/mL of ampicillin were added. Activity of toluene dioxygenase was induced by addition of 1 mM of IPTG at the same time. The culture was grown for another 2 hours to OD₆₀₀ 3.38. After that, air flow was reduced to 1 L/min. Additional glucose was added three times, 20 mM each addition at 5, 7 and 8.5 hours after inoculation. A total supply of 8 mL p-xylene was provided in small portions of 0.4 mL, each injected with a syringe directly to the fermentor over 4 hours every 10-15 min, beginning at 5 hours after inoculation. The culture was harvested after 12 hours of incubation (final OD₆₀₀ approximately 3.8), and cells were removed by centrifugation (15 min at 5000×g). To the clear yellowish supernatant, 280 g of NaCl were added and completely dissolved. The solution (˜1.6 L) was extracted 3 times×0.8 L of ethyl acetate. The solvent was evaporated under reduced pressure to give 1.53 g of the cis-glycol compound as nearly colorless crystals, homogenous on TLC analysis (silica gel plate, Rf˜0.4 in ethyl acetate, UV absorbing at 254 nm, positive for iodine vapor stain).

In another embodiment, the preparation was performed as above, except for the following: 2 molar equivalents of glycerol were used instead of every molar equivalent of glucose; an additional 60 μM ferrous ammonium sulfate were supplied at the time of induction with IPTG; addition of sodium chloride prior to extraction was omitted; and extraction was performed 4 times×0.6 L of ethyl acetate. After removal of ethyl acetate under reduced pressure, 3.30 g of essentially pure glycol (TLC) was obtained.

### Example 2

### Biocatalytic Oxidation of p-xylene

Changes (as opposed to the conditions in Example 1) in host strain, expression systems, and/or fermentor conditions are optionally implemented, e.g., to optimize production of cis-diol-dienes. The following example provides alternative conditions for enzymatically producing diol-dienes from substituted benzenes. Other conditions are also optionally used.

In an aerated agitated fermentor, 1.0 L of autoclaved minimal medium containing 3.5 g of NaNH₄HPO₄ 4H₂O, 7.5 g of K₂HPO₄ 3H2O, and 3.7 g of KH₂PO₄ (See, e.g., Lageveen, R. G., G. W. Huisman, H. Preusting, P. Ketelaar, G. Eggink, and B. Witholt (1988) Formation of polyesters by Pseudomonas oleovorans: effect of substrates on formation and composition of poly-(R)-3-hydroxyalkanoates and poly-(R)-3-hydroxyalkanoates, Appl. Environ. Microbiol. 54:2924-2932) pH 7.0, 5 ml of R₂ trace elements (See, e.g., Riesenberg, D., K. Menzel, V. Schulz, K. Schumann, G. Veith, G. Zuber, and W. A. Knorre (1990) High cell density fermentation of recombinant Escherichia coli expressing human interferon alpha 1, Appl. Microbiol. Biotechnol. 34:77-82), 20 ml of 10% yeast extract solution in water, 5 ml 1 M MgSO₄, 10 ml of 50% fructose solution in water were added. The temperature of the medium was brought to 37° C.

Ampicillin was added from a concentrated stock solution, to a final concentration of 100 μg/ml. The fermentor was inoculated with an overnight culture of Escherchia coli LS5218 (pTrctodNK1) grown in a shake flask containing the above minimal medium with R₂ trace element solution, pH 7.0, 0.1% yeast extract, 20 mM glucose, and 100 μg/ml ampicillin to a starting optical density at 600 nm (OD₆₀₀) of 0.22. The plasmid pTrctodNK1 was constructed by amplifying the todC1C2AB genes from Pseudomonas putida F1 (ATCC 700007) using the polymerase chain reaction (PCR) and cloning them into expression vector, pTrc99a (Amersham Pharmacia Biotech, Piscataway, N.J.). The air was supplied to the fermentation vessel at 1.8 L/min, the pH of the culture was maintained using a concentrated solution of potassium hydroxide in water. Ampicillin was added hourly to the fermentor at a final concentration of 100 μg/ml. The culture was grown to an OD₆₀₀ of 3.5, at which time a feed solution of 50% fructose, 6% ammonium chloride and 2% magnesium sulfate was initiated at a rate that resulted in a final concentration of 5% fructose in the culture. The dissolved oxygen was maintained about 30%. After the culture reached an OD₆₀₀ of 15, the temperature was reduced to 34° C. and 200 μM ferrous ammonium sulfate was added. The expression of toluene dioxygenase was induced by the addition of 250 μM of IPTG. The culture was grown for another 2 hours. After that, p-xylene was fed to the culture through the air stream at a flow rate of 3 L/min. The formation of the cis-diol-diene compound, 1,2-dihydroxy-3,6-dimethylhexa-3,5-diene (Formula 4), was monitored at regular intervals by measuring the absorbance of cell-free supernatant at a wavelength of 280 nm and estimating the concentration using an extinction coefficient of ε=6500 mol⁻¹ cm⁻¹ (See, e.g., and Gibson, D. T., V. Mahadevan, and J. F. Davey (1974) Bacterial metabolism of para- and meta-xylene: oxidation of the aromatic ring, J. Bacteriol. 119:930-936.). The amount of cis-diol-diene produced was 20 g/L.

### Example 3

### Protection of xylene-cis-diol to Form a Protected Diol-diene

1.0 g of cis-1,2-dihydroxy-3,6-dimethylhexa-3,5-diene (Formula 4) was dissolved in 10 ml of 2,2-dimethoxypropane, 5 ml of n-hexane and a crystal (−2 mg) of p-toluenesulfonic acid hydrate were added. The solution was stirred for 20 min at room temperature. 2 mL of potassium 0.5 M phosphate buffer pH 7.5 were added, and the reaction mixture was stirred for another 3 min. The aqueous lower layer was removed by a pipette, and the solution was dried by addition of 3 g of anhydrous sodium sulfate, filtered, and the solvent was removed under reduced pressure to yield 1.17 g of the acetonide represented by Formula (8) as clear colorless oil (91%) essentially pure on TLC analysis (silica gel plate, Rf˜0.7 in methylene chloride, UV absorbing at 254 nm, positive for iodine vapor staining).

### Example 4

### Chemical Oxidation of a Diol-diene to Form a Diol-dione

3,4-cis-isopropylidenedioxy-hexane-2,5-dione, (a compound represented by Formula (12)) was prepared by oxidation with permanganate/periodate of a compound having Formula (8). KMnO₄ (1.400 g) and MgSO₄ (600 mg) were dissolved in 15 ml of water, cooled to 0° C. and the resulting solution was added dropwise over 20 min to a cooled stirred solution of 500 mg of acetonide of cis-1,2-dihydroxy-3,6-dimethylhexa-3,5-diene in a mixture of 10 ml of methanol, and 20 ml of water. The reaction mixture was stirred for 20 min while the temperature was maintained at 0-4° C. NaIO₄ (2.200 g) was dissolved in 20 ml of water, cooled to 0° C., and the solution was added to the stirred reaction mixture dropwise over period of 15 minutes. The reaction temperature was raised to 20° C., and the stirring was continued for 8 hours. At the end of the reaction the pH of the mixture was about 4-5. The resulting yellowish solution was filtered, and 1 g NaHSO₃ was added and dissolved. Extraction with ethyl acetate (5×30 ml), drying over anhydrous Na₂SO₄ and evaporation furnished 330 mg (63.8%) of the dione of Formula (12) as a clear colorless oil, essentially pure on TLC analysis (silica gel plate, Rf˜0.6 in methylene chloride:ethyl acetate 4:1, no UV absorption at 254 nm, weakly positive for iodine vapor staining).

### Example 5

### Deprotection and Cyclization

Deprotection of 3,4-cis-isopropylidenedioxy-hexane-2,5-dione (Formula (12)) to 3,4-cis-dihydroxy-hexane-2,5-dione (Formula 6)) was performed using 5 ml of 0.05M H₂SO₄ in water, which was added to 100 mg of 3,4-isopropylidenedioxy-hexane-2,5-dione. The mixture was heated at 50° C. under nitrogen, with stirring for 3 hours. A 0.5 ml aliquot of the reaction mixture was extracted 5×1 ml of ethyl acetate, and after solvent evaporation yielded 7.3 mg (93%) of white crystalline solid 3,4-cis-dihydroxy-hexane-2,5-dione, which was essentially pure on TLC analysis (silica gel plate, Rf˜0.2 in ethyl acetate:methanol 10:1, no UV absorption at 254 nm, weakly positive for iodine vapor staining).

The pH of the remaining solution was adjusted with sodium hydroxide to about 9, and when the solution was heated under reflux, evolution of strong and unmistakable characteristic odor of the 4-hydroxy-2,5-dimethyl-2,3-dihydrofuran-3-one was observed.

### Example 6

### Oxidation of an unprotected diol-diene by ozonolysis to form a diol-dione

Oxidation of an unprotected diol-diene by ozonolysis to form a diol-dione was achieved using 1,2-dihydroxy-3,6-dimethylhexa-3,5-diene (Formula 4) also called p-xylene cis-2,3-dihydro-2,3-diol (PXD) prepared as described above and not purified prior to ozonolysis. All reagents were purchased from Fisher Scientific (Pittsburg, Pa.) and used without further purification.

Sodium sesquicarbonate solution A was prepared by dissolving 84 g of NaHCO₃ and 53 g of Na₂CO₃ in 1 L of water (heating is required to achieve complete dissolution). The final pH of the solution was about 10-11.

5.07 g of vacuum dried PXD was dissolved in 50 ml of MeOH and ozonized at −78° C. for 3 hrs until blue color persisted (65 kV, 31 pm). The resulting solution was transferred via insulated canula to a solution of 21 ml of 1M Na₂S₂O₃ and 13 ml of 1.5 M sesquicarbonate solution A, stirred at 0° C. The resulting solution quickly became peroxide negative. Stirring was continued for 30 min at 0° C. and for another 30 minutes at room temperature.

MeOH was distilled off under reduced pressure (40° C. on rotovap), and the resulting solution (with white precipitate) was extracted with EtOAc (4×125 ml). Combined extracts were dried over anhydrous sodium sulfate and evaporated to give colorless and practically odorless oil, which after vacuum drying partially crystallized on standing (4.92 g).

This method furnished the known 2,5-diketo-3,4-dihydroxyhexane (DD) (Formula 6) and an equal amount of a methoxyketal-hemiketal by-product (X), with combined yields of about 84-92%.

### Example 7

### Cyclization of an Unprotected Diol-dione Compound

The following conditions were used to prepare a furanone from an unprotected diol-dione, e.g., using a modification of a procedure published in U.S. Pat. No. 5,149,840, which describes and claims an optimized cyclization of rhamnose and other 6-deoxyhexoses to 4-hydroxy-2,5-dimethyl-2,3-dihydrofuran-3-one (Formula 1) (SF).

About 800 mg of crude ozonolysis product (DD+X, ˜1:1), as prepared above, was added to a buffer comprising: 340 mg of NaH₂PO₄×1 H₂O, 80 mg NaHCO3, 0.260 mL of H₂O, and 0.100 mL of 40% NaOH. The semi-solid mixture of crude DD+X was added to a solvent, e.g., either n-butylacetate (nBuOAc) or ethylacetate (EtOAc), and the two-phase system was heated, after degassing with argon, in a closed vial (see Table 2). The solvent was separated from the dark-reddish-brown aqueous phase, the aqueous layer was extracted with ethylacetate (3×5 ml) and the organic layer and extracts were combined, dried over anhydrous MgSO₄ and evaporated on a rotovap. All procedures were repeated at least 3 times, which were reproducible up to 2-6 g.

TABLE 2
Summary of various conditions used for two-phase cyclization
reactions to produce 4-hydroxy-3[2H]-furanone

| | | Buffer | | | | |
|---|---|---|---|---|---|---|
| Weight, | | amt. of | | | | Ratio of |
| starting | | NaH₂PO₄ × 1 | | | | Furanone to |
| Material | | H₂O | Temp., | Time | Weight | diol-dione |
| DD + X | Solvent | is given | ° C. | (hrs) | recovered | (by NMR) |
| 800 mg | nBuOAc, | 340 mg + | 95, | 4 | 280 mg | 4:1 |
| | 3 mL | 80 mg | then r.t. | 16 | | |
| | | NaHCO₃ | | | | |
| 1.3 g | EtOAc | 510 mg + | Reflux | 22 | 630 mg | 1:1 |
| | 3 mL | 120 mg | (˜80° C.) | | | |
| | | NaHCO₃ | | | | |
| 1 g | nBuOAc, | 425 mg + | 95, | 8 | 390 mg | 3.5:1 |
| | 5 mL | 100 mg | then r.t. | 15 | | |
| | | NaHCO₃ | | | | |
| 2.6 g | nBuOAc, | 170 mg + | 110 | 22 | 1.3 g | 3:1 |
| | 10 mL | 40 mg | | | | |
| | | NaHCO₃ | | | | |

The material was analyzed by TLC, producing one major spot, and by ¹H NMR (in comparison to a standard comprising Formula 1), with deuterochloroform as solvent (D₂O exchange was also done). The final 4-hydroxy-2,5-dimethyl-2,3-dihydrofuran-3-one (Formula 1) product is very volatile and oxidizes readily in the presence of air, as evidenced by appearance of additional spots on TLC.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patent applications, patents, and other documents cited in this application are incorporated by reference in their entirety for all purposes to the same extent as if each individual publication or patent document were individually so denoted.

What is claimed is:

1. A method of making a 4-hydroxy-3[2H]-furanone, the method comprising:
(i) providing a substituted benzene;
(ii) enzymatically oxidizing the substituted benzene, thereby producing a cis-diol-diene compound;
(iii) oxidizing the diol-diene compound, thereby forming a cis-diol-dione compound; and,
(iv) cyclizing the diol-dione compound, thereby making a 4-hydroxy-3[2H]-furanone.

2. The method of claim 1, wherein the 4-hydroxy-3[2H]-furanone comprises 4-hydroxy-2,5-dimethyl-3[2H]-furanone.

3. The method of claim 1, wherein the 4-hydroxy-3[2H]-furanone comprises a compound having formula 1: 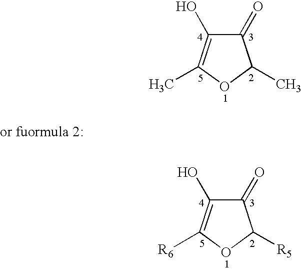
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

4. The method of claim 3, wherein R₅ and R₆ are not both hydrogen.

5. The method of claim 3, wherein the lower alkyl comprises methyl, ethyl, propyl, isopropyl, isobutyl, sec-butyl, or tert-butyl.

6. The method of claim 1, wherein the substituted benzene comprises p-xylene.

7. The method of claim 1, wherein the substituted benzene comprises a compound having formula 3: 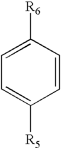
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

8. The method of claim 7, wherein R₅ and R₆ are not both hydrogen.

9. The method of claim 7, wherein the lower alkyl comprises methyl, ethyl, propyl, isopropyl, isobutyl, sec-butyl, or tert-butyl.

10. The method of claim 1, step (ii) producing a compound of formula 4: 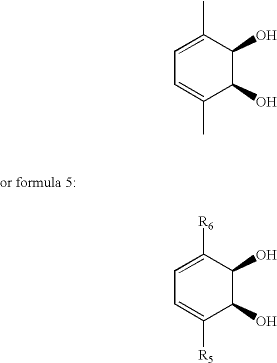
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

11. The method of claim 10, wherein R₅ and R₆ are not both hydrogen.

12. The method of claim 10, wherein the lower alkyl comprises methyl, ethyl, propyl, isopropyl, isobutyl, sec-butyl, or tert-butyl.

13. The method of claim 1, wherein the cis-diol-diene compound comprises an achiral diol-diene.

14. The method of claim 1, wherein the cis-diol-diene compound comprises a chiral cis-diol-diene compound.

15. The method of claim 1, wherein the cis-diol-diene compound comprises cis-1,2-dihydroxy-3,6-dimethylhexa-3,5-diene.

16. The method of claim 1, wherein step (ii) comprises contacting the substituted benzene with a dioxygenase.

17. The method of claim 16, wherein the dioxygenase comprises an arene dioxygenase.

18. The method of claim 16, wherein the dioxygenase is selected from one or more of toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene 1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, and ring dihydroxylating dioxygenase.

19. The method of claim 16, wherein the dioxygenase comprises a toluene dioxygenase, a tetrachlorobenzene dioxygenase, or an isopropylbenzene dioxygenase.

20. The method of claim 16, wherein the dioxygenase comprises a toluene dioxygenase.

21. The method of claim 16, wherein the dioxygenase is encoded by a nucleic acid comprising a mutant or chimeric dioxygenase nucleotide sequence.

22. The method of claim 21, wherein the dioxygenase is encoded by a nucleic acid comprising a mutant or chimeric arene dioxygenase nucleotide sequence.

23. The method of claim 21, wherein the nucleic acid comprises a polynucleotide sequence comprising at least 60 contiguous nucleotides of a nucleic acid encoding one or more of: toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, ring dihydroxylating dioxygenase, and any arene dioxygenase that is present in a public database such as GenBank™ at the time of filing of the subject application.

24. The method of claim 21, wherein the nucleic acid encodes a polypeptide having at least 20 contiguous amino acids of one or more of: toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, ring dihydroxylating dioxygenase, and any arene dioxygenase that is present in a public database such as GenBank™ at the time of filing of the subject application.

25. The method of claim 21 or claim 22, wherein the nucleic acid hybridizes under stringent conditions to at least one nucleic acid encoding a dioxygenase selected from toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, ring dihydroxylating dioxygenase, or any arene dioxygenase that is present in a public database such as GenBank™ at the time of filing of the subject application.

26. The method of claim 1, wherein step (ii) comprises contacting the substituted benzene with one or more cells, which cells possess dioxygenase activity.

27. The method of claim 26, wherein the cells are microbial cells.

28. The method of claim 27, wherein the cells are bacterial cells.

29. The method of claim 1, further comprising enzymatically oxidizing the substituted benzene in the presence of one or more of water and an organic solvent.

30. The method of claim 1, step (iii) forming a compound having formula 6 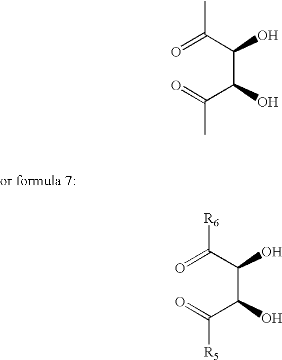
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

31. The method of claim 30, wherein R₅ and R₆ are not both hydrogen.

32. The method of claim 30, wherein the lower alkyl comprises methyl, ethyl, propyl, isopropyl, isobutyl, sec-butyl, or tert-butyl.

33. The method of claim 1, wherein the diol-dione compound comprises hexane-3,4-cis-diol-2,5-dione.

34. The method of claim 1, wherein step (iii) comprises:
(a) protecting a first hydroxyl group and a second hydroxyl group of the cis-diol-diene compound, thereby producing a protected cis-diol-diene compound;
(b) oxidizing the protected cis-diol-diene compound, thereby forming a protected dione compound; and
(c) deprotecting the protected dione compound, thereby providing the cis-diol-dione compound.

35. The method of claim 34, wherein the protected cis-diol-diene compound comprises an achiral compound.

36. The method of claim 34, wherein step (a) comprises forming a cyclic ketal, a cyclic acetal, an ether group, or an ester group.

37. The method of claim 36, wherein forming the cyclic ketal or the cyclic acetal results in a compound having formula 8: 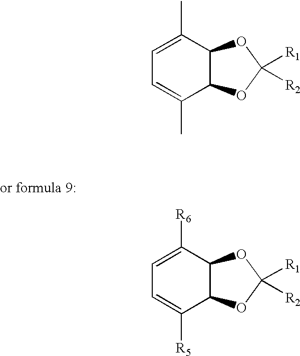
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl; and
wherein R₁ and R₂ are each independently selected from: hydrogen, alkyl, aryl, and aralkyl or R₁ and R₂ together comprise a cycloalkyl ring, which cycloalkyl ring comprises about 5 to about 6 carbon atoms.

38. The method of claim 37, wherein R₅ and R₆ are not both hydrogen.

39. The method of claim 37, wherein the lower alkyl comprises methyl, ethyl, propyl, isopropyl, isobutyl, sec-butyl, or tert-butyl.

40. The method of claim 37, wherein R₁ and R₂ are the same or different.

41. The method of claim 37, wherein at least one of R₁ and R₂ is not hydrogen.

42. The method of claim 36, wherein forming the ether group or the ester group results in a compound having formula 10: 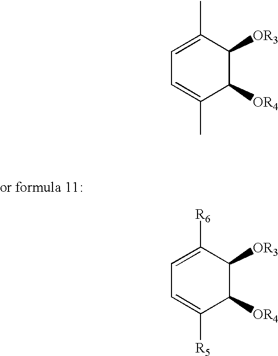
wherein R₃ and R₄ are independently selected from: hydrogen, alkylacyl, arylacyl, tert-butyl, trialkylsilyl, and aralkylacyl; or R₃ and R₄ together comprise a boron moiety having an alkyl, aryl, or hydroxy substituent; and,
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

43. The method of claim 42, wherein R₅ and R₆ are not both hydrogen.

44. The method of claim 42, wherein the lower alkyl comprises methyl, ethyl, propyl, isopropyl, isobutyl, sec-butyl, or tert-butyl.

45. The method of claim 34, wherein step (a) comprises contacting the cis-diol-diene compound with one or more ketone or ketal.

46. The method of claim 45, further comprising contacting the cis-diol-diene compound with one or more ketone or ketal in the presence of a catalyst.

47. The method of claim 46, wherein the catalyst comprises an acid catalyst.

48. The method of claim 47, wherein the acid catalyst comprises aryl or alkylsulfonic acid.

49. The method of claim 46, wherein the catalyst comprises a solid phase catalyst.

50. The method of claim 49, wherein the solid phase catalyst comprises a solid phase acid.

51. The method of claim 46, wherein the catalyst comprises a resin, which resin comprises protonated sulfonic groups.

52. The method of claim 34, wherein step (b) comprises contacting the protected diol-diene compound with one or more oxidizing reagent.

53. The method of claim 34, wherein step (b) results in a compound having 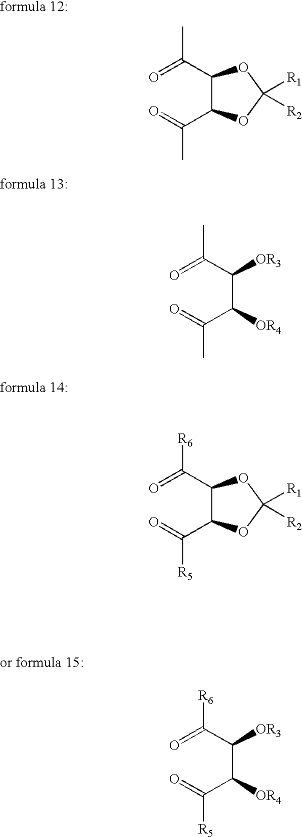
wherein R₁ and R₂ are each independently selected from: hydrogen, alkyl, aryl, and aralkyl or wherein R₁ and R₂ together comprise a cycloalkyl ring, which cycloalkyl ring comprises about 5 to about 6 carbon atoms;
wherein R₃ and R₄ are independently selected from: hydrogen, alkylacyl, arylacyl, tert-butyl, trialkylsilyl, and aralkylacyl; or R₃ and R₄ together comprise a boron compound having an alkyl, aryl or hydroxy substituent; and,
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

54. The method of claim 53, wherein R₅ and R₆ are not both hydrogen.

55. The method of claim 53, wherein the lower alkyl comprises methyl, ethyl, propyl, isopropyl, isobutyl, sec-butyl, or tert-butyl.

56. The method of claim 52, wherein the one or more oxidizing reagent comprises one or more of: an alkali metal salt, an alkali metal permanganate salt, an alkali metal periodate salt, an alkali metal hypochlorite salt, an organic peroxyacid, an organic peroxide, an inorganic peroxyacid, an inorganic peroxide, ozone, and an ozone/oxygen mixture.

57. The method of claim 52, comprising contacting the protected diol-diene compound with an alkali metal hypochlorite salt in the presence of catalytic amounts of ruthenium halide or oxide.

58. The method of claim 34, wherein step (c) comprises contacting the protected dione compound with one or more deprotecting reagent.

59. The method of claim 58, wherein the protected dione compound comprises a cyclic ketal or a cyclic acetal and the one or more deprotecting reagent comprises acetic acid, hydrochloric acid, sulfuric acid, phosphoric acid, oxalic acid, or citric acid.

60. The method of claim 34, step (c) providing a compound having formula 6: 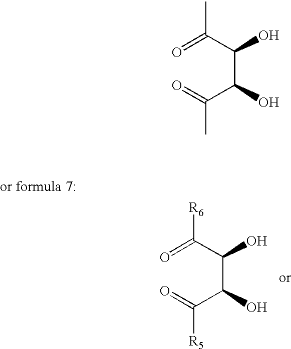
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

61. The method of claim 60, wherein R₅ and R₆ are not both hydrogen.

62. The method of claim 60, wherein the lower alkyl comprises methyl, ethyl, propyl, isopropyl, isobutyl, sec-butyl, or tert-butyl.

63. The method of claim 1, wherein step (iii) comprises oxidizing the cis-diol-diene compound in a substantially aqueous solvent comprising ozone or a mixture of ozone and oxygen in the presence of boric acid, arylboronic acid, alkyl boronic acid, or a metal salt thereof.

64. The method of claim 63, further comprising attaching the cis-diol-diene compound to a resin or inorganic adsorbent material, which resin or inorganic adsorbent material comprises an alkylboronate moiety or an arylboronate moiety.

65. The method of claim 1, wherein step (iv) comprises cyclizing the diol-dione compound in the presence of a catalyst or an amino acid.

66. The method of claim 65, wherein the catalyst comprises an alkali metal salt of a dibasic or tribasic acid or an alkali-earth metal salt of a dibasic or tribasic acid.

67. The method of claim 1, comprising isolating the cis-diol-dione compound.

68. The method of claim 1, comprising performing step (iii) and step (iv) contemporaneously and thereby cyclizing the cis-diol-dione compound in an unisolated format.

69. The method of claim 3, 7, 10, 30, 37, 42, 53, or 60 wherein
R₅ is hydrogen and R₆ is selected from: lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl and geminal dialkoxyalkyl; or
R₅ is methyl and R₆ is selected from: ethyl, propyl, isopropyl, acetyl, propanoyl, 1-hydroxyethyl, and 2-hydroxyethyl; or
R₅ is ethyl and R₆ is selected from ethyl, acetyl, and propanoyl.

70. The method of claim 69, wherein the lower alkyl comprises isopropyl, isobutyl, sec-butyl, or tert-butyl.

71. The method of claim 3, 7, 10, 30, 37, 42, 53, or 60 wherein the lower alkyl comprises an alkyl comprising about 1 to about 6 carbon atoms.

72. The method of claim 3, 7, 10, 30, 37, 42, 53, or 60 wherein at least one of R₅ and R₆ comprises two or more carbons.

73. The method of claim 3, 7, 10, 30, 37, 42, 53, or 60 wherein R₅ or R₆ comprises a methyl group.

74. The method of claim 3, 7, 10, 30, 37, 42, 53, or 60 wherein R₅ and R₆ are different and wherein at least one of R₅ or R₆ comprises two or more carbon atoms.

75. A composition comprising a compound having formula 12: 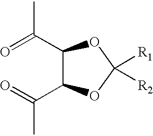
wherein R₁ and R₂ are each independently selected from: hydrogen, alkyl, aryl, and aralkyl or wherein R₁ and R₂ together comprise a cycloalkyl ring comprising about 5 to about 6 carbon atoms.

76. The composition of claim 75, the compound of formula 12 comprising substantially all cis-stereoisomers.

77. A composition comprising a compound having formula 13: 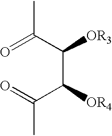
wherein R₃ and R₄ are independently selected from: hydrogen, alkylacyl, arylacyl, tert-butyl, trialkylsilyl, and aralkylacyl.

78. The composition of claim 77, the compound of formula 13 comprising substantially all cis-stereoisomers.

79. A composition comprising a compound having formula 14: 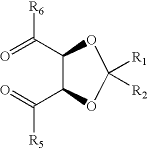
wherein R₁ and R₂ are each independently selected from: hydrogen, alkyl, aryl, and aralkyl or wherein R₁ and R₂ together comprise a cycloalkyl ring comprising about 5 to about 6 carbon atoms; and,
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

80. The method of claim 79, wherein the lower alkyl comprises methyl, ethyl, propyl, isopropyl, isobutyl, sec-butyl, or tert-butyl.

81. The method of claim 79, wherein R₅ and R₆ are not both hydrogen.

82. The composition of claim 79, the compound of formula 14 comprising substantially all cis-stereoisomers.

83. A composition comprising a compound having formula 15: 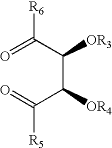
wherein R₃ and R₄ are independently selected from: hydrogen, alkylacyl, arylacyl, tert-butyl, trialkylsilyl, and aralkylacyl; or R₃ and R₄ together comprise a boron compound having an alkyl, aryl or hydroxy substituent; and,
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

84. The method of claim 83, wherein the lower alkyl comprises methyl, ethyl, propyl, isopropyl, isobutyl, sec-butyl, or tert-butyl.

85. The method of claim 83, wherein R₅ and R₆ are not both hydrogen.

86. The composition of claim 83, the compound of formula 15 comprising substantially all cis-stereoisomers.

87. A composition comprising a compound having formula 7: 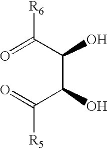
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

88. The method of claim 87, wherein the lower alkyl comprises methyl, ethyl, propyl, isopropyl, isobutyl, sec-butyl, or tert-butyl.

89. The method of claim 87, wherein R₅ and R₆ are not both hydrogen.

90. The composition of claim 90, the compound of formula 7 comprising substantially all cis-stereoisomers

91. A composition comprising a compound having formula 2: 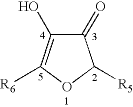
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

92. The method of claim 91, wherein the lower alkyl comprises methyl, ethyl, propyl, isopropyl, isobutyl, sec-butyl, or tert-butyl.

93. The method of claim 91, wherein R₅ and R₆ are not both hydrogen.

94. A composition comprising at least 0.1 ppm of one or more of the compounds of claim 91.

95. The composition of claim 94, the composition comprising a flavoring composition, a food flavoring compositions, a beverage flavoring composition, an odor control composition or a laundry composition.

96. The method of claim 1, comprising enzymatically oxidizing the substituted benzene with an enzyme produced by a method comprising:
(a) providing a population of DNA fragments, which DNA fragments encode at least one parental enzyme, which at least one parental enzyme oxidizes a substituted benzene;
(b) recombining the DNA fragments to produce a library of recombinant DNA segments;
(c) optionally repeating the recombination of steps (i) and (ii);
(d) screening the library of recombinant DNA segments to identify at least one recombinant DNA segment that encodes an artificially evolved enzyme, which artificially evolved enzyme comprises greater oxidizing activity for substituted benzenes than that encoded by the parental enzyme; and,
(e) repeating steps (i) through (iv) one or more times.

97. The method of claim 96, wherein the oxidizing activity is selected from conversion rate and substrate specificity.

98. The method of claim 96, wherein the at least one parental enzyme is selected from: toluene dioxygenase, tetrachlorobenzene dioxygenase, 1,2,4-trichlorobenzene dioxygenase, ethylbenzene dioxygenase, chlorobenzene dioxygenase, benzene dioxygenase, isopropylbenzene dioxygenase, biphenyl dioxygenase, indene1,2-dioxygenase, napthalene dioxygenase, 2-nitrotoluene 2,3-dioxygenase, 2,4-dinitrotoluene dioxygenase, phenanthrene dioxygenase, phenylproprionate 2,3-dioxygenase, cinnimate 2,3-dioxygenase, 2-halobenzoate 1,2-dioxygenase, ortho-halobenzoate 1,2-dioxygenase, anthranilate dioxygenase, m,p-toluate 1,2 dioxygenase, p-cumate 2,3-dioxygenase, 3(4)-phenoxybenzoate 3,4-dioxygenase, phthalate 3,4-dioxygenase, diterpenoid ring dihydroxylating dioxygenase, diterpenoid ring hydroxylating dioxygenase, aniline 1,2-dioxygenase, carbazole dioxygenase, ring dihydroxylating dioxygenase, or any arene dioxygenase that is present in a public database such as GenBank™ at the time of filing of the subject application.

99. The method of claim 96, wherein the artificially evolved enzyme oxidizes a substituted benzene having formula 3: 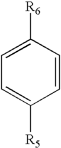
to form a diol-diene compound having formula 5: 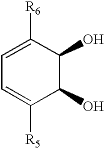
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

100. The method of claim 99, wherein the lower alkyl comprises methyl, ethyl, propyl, isopropyl, isobutyl, sec-butyl, or tert-butyl.

101. The method of claim 99, wherein R₅ and R₆ are not both hydrogen.

102. The method of claim 99, wherein
R₅ is hydrogen and R₆ is selected from: lower alkyl, isopropyl, isobutyl, sec-butyl, tert-butyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, and geminal dialkoxyalkyl; or
R₅ is methyl and R₆ is selected from: ethyl, propyl, isopropyl, acetyl, propanoyl, 1-hydroxyethyl, and 2-hydroxyethyl; or
R₅ is ethyl and R₆ is selected from ethyl, acetyl, and propanoyl.

103. The method of claim 99, wherein the lower alkyl comprises an alkyl comprising about 1 to about 6 carbon atoms.

104. The method of claim 99, wherein at least one of R₅ and R₆ comprises two or more carbons.

105. The method of claim 99, wherein R₅ or R₆ is a methyl group.

106. The method of claim 99, wherein R₅ and R₆ are different and wherein at least one of R₅ or R₆ comprises two or more carbon atoms.

107. The method of claim 96, wherein the artificially evolved enzyme oxidizes p-xylene to form cis-1,2-dihydroxy-3,6-dimethylhexa-3,5-diene.

108. A nucleic acid library produced by the method of claim 96.

109. A population of cells comprising the library of claim 108.

110. A recombinant dioxygenase homologue produced by the method of claim 96.

111. A cell comprising the dioxygenase homologue of claim 110.

112. A composition comprising an artificially evolved enzyme of claim 96 and a substituted benzene, wherein the substituted benzene comprise p-xylene or a compound having formula 3: 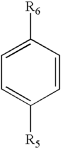
wherein R₅ and R₆ are independently selected from: hydrogen, lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl, geminal dialkoxyalkyl, acetyl, and propanoyl.

113. The method of claim 112, wherein the lower alkyl comprises methyl, ethyl, propyl, isopropyl, isobutyl, sec-butyl, or tert-butyl.

114. The method of claim 112, wherein R₅ and R₆ are not both hydrogen.

115. The composition of claim 79, 83, 87, 91, or 112, wherein
R₅ is hydrogen and R₆ is selected from: lower alkyl, cyclohexyl, phenyl, benzyl, methoxymethyl, ethoxymethyl, 2-methoxymethyl, 2-hydroxy-2-propyl, 2-hydroxy-1-propyl, 1-hydroxyethyl, 2-hydroxyethyl, 2-keto-1-propyl, 2-keto-1-butyl, 3-keto-1-butyl and geminal dialkoxyalkyl; or
R₅ is methyl and R₆ is selected from: ethyl, propyl, isopropyl, acetyl, propanoyl, 1-hydroxyethyl, and 2-hydroxyethyl; or
R₅ is ethyl and R₆ is selected from ethyl, acetyl, and propanoyl.

116. The method of claim 115, wherein the lower alkyl comprises isopropyl, isobutyl, sec-butyl, or tert-butyl.

117. The composition of claim 79, 83, 87, 91, or 112, wherein the lower alkyl comprises an alkyl comprising about 1 to about 6 carbon atoms.

118. The composition of claim 79, 83, 87, 91, or 112, wherein at least one of R₅ and R₆ comprises two or more carbons.

119. The composition of claim 79, 83, 87, 91, or 112, wherein R₅ or R₆ is a methyl group.

120. The composition of claim 79, 83, 87, 91, or 112, wherein R₅ and R₆ are different and wherein at least one of R₅ or R₆ comprises two or more carbon atoms.
